(19) United States
(12) Patent Application Publication
(10) Pub. No.: US 2024/0010726 A1
(43) Pub. Date: Jan. 11, 2024

(54) LFA-1 SIGNALLING MEDIATOR FOR USE IN CANCER THERAPY

(71) Applicant: Universität Basel, Basel (CH)

(72) Inventors: Jonas LÖTSCHER, Binningen (CH); Christoph HESS, Basel (CH)

(21) Appl. No.: 18/021,323

(22) PCT Filed: Aug. 17, 2021
(86) PCT No.: PCT/EP2021/072865

§ 371 Date: Feb. 14, 2023

(30) Foreign Application Priority Data
Aug. 17, 2020 (EP) 20191392.8

(51) Int. Cl. C07K 16/28; A61P 35/00; A61K 9/00

(52) U.S. Cl. CPC C07K 16/2818; A61P 35/00; A61K 9/0019

(57) ABSTRACT

The present invention relates to an LFA-1 signalling mediator with moderate LFA-1 stabilization properties for use in cancer immunotherapy or a composition for use in cancer immunotherapy comprising an immune system modulator, wherein the immune system modulator enhances the immune response against cancer, and an LFA-1 signalling mediator with moderate LFA-1 stabilization properties wherein the LFA-1 signalling mediator selectively and significantly enhances the anti-cancer immune response. The composition may comprise a carrier for target delivery of the composition.

The present invention relates to an LFA-1 signalling mediator with moderate LFA-1 stabilization properties for use in cancer immunotherapy or a composition for use in cancer immunotherapy comprising an immune system modulator, wherein the immune system modulator enhances the immune response against cancer, and an LFA-1 signalling mediator with moderate LFA-1 stabilization properties wherein the LFA-1 signalling mediator selectively and significantly enhances the anti-cancer immune response. The composition may comprise a carrier for target delivery of the composition.

Surgery, radiation therapy, and chemotherapy are the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed. As key immunoregulatory molecules and signals of immunity are identified and prepared as therapeutic reagents, the clinical effectiveness of such reagents can be tested using well-known cancer models. Immunotherapeutic strategies include administration of vaccines, activated cells, antibodies, cytokines, chemokines, as well as small molecular inhibitors, anti-sense oligonucleotides, and gene therapy (Mocellin, et al., Cancer Immunol. & Immunother. (2002) 51: 583-595; Dy, et al., J. Clin. Oncol. (2002) 20: 2881-2894, 2002).

The growth and metastasis of cancer and tumors depend to a large extent on their capacity to evade host immune surveillance and overcome host defenses. Most cancer and tumors express antigens that can be recognized to a variable extent by the host immune system, but in many cases, the immune response is inadequate. Failure to elicit a strong activation of effector T cells may result from the weak immunogenicity of tumor antigens or inappropriate or absent expression of co-stimulatory molecules by cancer and tumor cells (Epstein, A., & Hu, P. (2012). U.S. Pat. No. 8,268,788. Washington, DC: U.S. Patent and Trademark Office). Leukocyte function-associated antigen (LFA-1, alphaLbeta2, CD11a/CD18) is an integrintype cell adhesion molecule that is predominantly involved in leukocyte trafficking and extravasation. LFA-1 is expressed on leukocytes and interacts with ligands ICAM-1, ICAM2, and ICAM-3 to promote a variety of homotypic and heterotypic cell adhesion events required for functions of the immune system, such as cell-cell, cell-matrix and cell-pathogen interactions (Arnaout M A. Integrin structure: new twists and turns in dynamic cell adhesion. Immunol Rev. 2002; 186:125-40; Askari J A, Buckley P A, Mould A P, Humphries M J.

Linking integrin conformation to function. J Cell Sci. 2009; 122:165-70; Caswell P T, Norman J C. Integrin trafficking and the control of cell migration. Traffic. 2006; 7:14-21; Huttenlocher A, Sandborg R R, Horwitz A F. Adhesion in cell migration. Curr Opin Cell Biol. 1995; 7:697-706; Huttenlocher A, Ginsberg M R, Horwitz A F. Modulation of cell migration by integrinmediated cytoskeletal linkages and ligand-binding affinity. J Cell Biol. 1996; 134:1551-62). LFA-1 is often expressed on the cell surface in an inactive state and mediates a low basal adhesiveness (Zhang K, Chen J. The regulation of integrin function by divalent cations. Cell Adh Migr. 2012; 6(1):20-29).

LFA-1-mediated adhesion and signalling events are important in normal physiological responses, including immune response (Springer T A, Wang J-h. The three-dimensional structure of integrins and their ligands and conformational regulation of cell adhesion. Adv Protein Chem. 2004; 68:29-63; Bon G, Folgiero V, Di Carlo S, Sacchi A, Falcioni R. Involvement of α6β4 integrin in the mechanisms that regulate breast cancer progression. Breast Cancer Res. 2007; 9:203; Di Sabatino A, Rovedatti L, Rosado M M, Carsetti R, Corazza G R, MacDonald T T. Increased expression of mucosal addressin cell adhesion molecule 1 in the duodenum of patients with active celiac disease is associated with depletion of integrin α4β7-positive T cells in blood. Hum Pathol. 2009; 40:699-704; Varner J A, Cheresh D A. Tumor angiogenesis and the role of vascular cell integrin αVβ3. Important Adv Oncol. 1996:69-87). The anatomy and binding sites of LFA-1 mediators have been described previously (Grönholm, Mikaela, et al. “LFA-1 integrin antibodies inhibit leukocyte α4β1-mediated adhesion by intracellular signalling.” Blood 128.9 (2016): 1270-1281; Zecchinon, Laurent, et al. “Anatomy of the lymphocyte function-associated antigen-1.” Clinical and Applied Immunology Reviews 6.3-4 (2006): 149-172.). Three conformational states of LFA-1 are known: the bent conformation with closed headpiece, the extended conformation with closed headpiece and the extended conformation with open headpiece, which are corresponding to the low-, intermediate- and high-affinity states, respectively (Takagi J, Petre B M, Walz T, Springer T A. Global conformational rearrangements in integrin extracellular domains in outside-in and inside-out signaling. Cell. 2002; 110:599-611; Zhang K, Chen J. The regulation of integrin function by divalent cations. Cell Adh Migr. 2012; 6(1):20-29). LFA-1 activation was described as being accompanied with a switchblade-like opening of the headpiecetailpiece interface, which extends the ligand-binding headpiece of the integrin heterodimer away from the plasma membrane (Takagi J, Petre B M, Walz T, Springer T A. Global conformational rearrangements in integrin extracellular domains in outside-in and inside-out signaling. Cell. 2002; 110:599-611; Zhang K, Chen J. The regulation of integrin function by divalent cations. Cell Adh Migr. 2012; 6(1):20-29). LFA-1 on the cell surface is in an equilibrium among these conformational states and may be stabilized in the active formation by LFA-1 signalling mediators with LFA-1 stabilization properties (Takagi J, Petre B M, Walz T, Springer T A. Global conformational rearrangements in integrin extracellular domains in outside-in and inside-out signaling. Cell. 2002; 110:599-611; Zhang K, Chen J. The regulation of integrin function by divalent cations. Cell Adh Migr. 2012; 6(1): 20-29).

LFA-1 can also be activated by antibodies, peptide, small molecule, divalent cations such as Mg²⁺ or Mn²⁺, or other stimuli (Zhang M., March M. E., Lane W. S., Long E. O. A signaling network stimulated by β2 integrin promotes the polarization of lytic granules in cytotoxic cells. Sci. Signal. 2014; 7:ra96; Traunecker E., Gardner R., Fonseca J. E., Polido-Pereira J., Seitz M., Villiger P. M., Iezzi G., Padovan E. Blocking of LFA-1 enhances expansion of Th17 cells induced by human CD14(+) CD16(+) nonclassical monocytes. Eur. J. Immunol. 2015; 45:1414-1425; Verma N. K., Fazil M. H., Ong S. T., Chalasani M. L. S., Low J. H., Kottaiswamy A., Praseetha P., Kizhakeyil A., Kumar S., Panda A. K., et al. LFA-1/ICAM-1 Ligation in Human T Cells Promotes Th1 Polarization through a GSK3β Signaling—Dependent Notch Pathway. J. Immunol. 2016; 197:108-118; Meli A. P., Fontés G., Avery D. T., Leddon S. A., Tam M., Elliot M., Ballesteros-Tato A., Miller J., Stevenson M. M., Fowell D. J., et al. The Integrin LFA-1 Controls T Follicular Helper Cell Generation and Maintenance. Immunity. 2016; 45:831-846; Gahmberg C. G., Fagerholm S. C., Nurmi S. M., Chavakis T., Marchesan S., Grönholm M. Regulation of integrin activity and signaling. Biochim. Biophys. Acta. 2009; 1790:431-444; Mócsai A., Walzog B., Lowell C. A. Intracellular signalling during neutrophil recruitment. Cardiovasc. Res. 2015; 107:373-385).

The affinity of the divalent cations to LFA-1 gradually decreases in the order of Mn²⁺>Mg²⁺>Ca²⁺ (Vorup-Jensen T, Waldron T T, Astrof N, Shimaoka M, Springer T A. The connection between metal ion affinity and ligand affinity in integrin I domains. Biochim Biophys Acta. 2007; 1774(9):1148-1155). The LFA-1 stabilization properties of antibodies can be stronger than the LFA-1 stabilization properties of divalent cations (Schürpf, Thomas, and Timothy A Springer. “Regulation of integrin affinity on cell surfaces.” The EMBO journal vol. 30, 23 4712-27. 23 Sep. 2011).

Modulation of integrins, such as LFA-1, in cancer immunotherapy remains complex. Previous research suggested beneficial effects from blockage of LFA-1 but, attempts to functionally antagonize integrins in human tumors have generally failed (Goodman, Simon L, and Martin Picard. “Integrins as therapeutic targets.” Trends in pharmacological sciences vol. 33,7 (2012): 405-12.). RGD-binding integrins were used as a target for antibody Fc effector functions in the context of cancer immunotherapy (Kwan, Byron H et al. “Integrin-targeted cancer immunotherapy elicits protective adaptive immune responses.” The Journal of experimental medicine vol. 214,6 (2017): 1679-1690). When targeting the integrin LFA-1 non-moderately, e.g. with antibodies, unforeseen effects when targeting LFA-1 are not uncommon (Reina, Manuel, and Enric Espel. “Role of LFA-1 and ICAM-1 in Cancer.” Cancers vol. 9,11 153. 3 Nov. 2017; Grönholm M, Jahan F, Bryushkova E A, et al. LFA-1 integrin antibodies inhibit leukocyte α4β1-mediated adhesion by intracellular signaling. Blood. 2016; 128(9):1270-1281).

In view of the above, there is an urgent need to selectively enhance cancer immunotherapy.

The above technical problem is solved by the embodiments provided herein and as characterized in the claims.

Accordingly, the present invention relates to the following embodiments.
1. A composition for use in cancer immunotherapy comprising
   (a) an immune system modulator, wherein the immune system modulator enhances the immune response against cancer, and
   (b) an LFA-1 signalling mediator with moderate LFA-1 stabilization properties wherein the LFA-1 signalling mediator significantly enhances the anti-cancer immune response.
2. An LFA-1 signalling mediator with moderate LFA-1 stabilization properties for use in cancer immunotherapy, wherein the LFA-1 signalling mediator enhances the anti-cancer immune response.
3. The composition for use of embodiment 1 or the LFA-1 signalling mediator for use of embodiment 2, wherein the LFA-1 signalling mediator induces selective T-cell mediated killing of cells presenting tumor-associated antigens.
4. The composition for use of embodiment 1, 3 or the LFA-1 signalling mediator for use of embodiment 2, 3, wherein the LFA-1 signalling mediator with moderate LFA-1 stabilization properties induces less T-cell mediated killing of cells not presenting tumor-associated antigens than a signalling mediator with strong LFA-1 stabilization properties.
5. The composition for use of embodiment 4, or the LFA-1 signalling mediator for use of embodiment 4, wherein the LFA-1 signalling mediator with strong LFA-1 stabilization properties is CBR LFA-1/2.
6. The composition for use of embodiment 1, 3-5 or the LFA-1 signalling mediator for use of embodiment 2-5, wherein the LFA-1 signalling mediator binds the metal-ion dependent adhesion site.
7. The composition for use of embodiment 1, 3-6, or the LFA-1 signalling mediator for use of embodiment 2-6, wherein the LFA-1 signalling mediator is a divalent cation.
8. The composition for use of embodiment 7, or the LFA-1 signalling mediator for use of embodiment 7, wherein the divalent cation is Mg²⁺.
9. The composition for use according to any one of embodiments 1, 3-8, wherein the immune system modulator is a monoclonal antibody, a modified immune cell or a checkpoint inhibitor (CPI).
10. The composition for use of embodiment 9, wherein the checkpoint inhibitor is a PD-1/PD-L1 inhibitor.
11. The composition for use of embodiment 10, wherein the PD-1/PD-L1 inhibitor is an inhibitor selected from the group consisting of nivolumab, pembrolizumab, cemiplimab, spartalizumab, atezolizumab, durvalumab and avelumab.
12. The composition for use according to any one of embodiments 1,3-11, or the LFA-1 signalling mediator for use of embodiments 2-8, 11, additionally comprising a carrier for targeted delivery of the LFA-1 signalling mediator.
13. The composition for use of embodiment 12 or the LFA-1 signalling mediator for use of embodiment 12, wherein the carrier is a membrane-forming molecule.
14. The composition for use of embodiment 13 or the LFA-1 signalling mediator for use of embodiment 13, wherein the membrane-forming molecule is a capsule-forming lipid.
15. The composition for use according to any one of embodiments 1, 3-14, wherein the immune system modulator and the LFA-1 signalling mediator are administered simultaneously or sequentially.
16. The composition for use of embodiment 15, wherein the immune system modulator and the LFA-1 signalling mediator are administered sequentially.
17. The composition for use of embodiment 16, wherein the immune system modulator is administered first, followed by the repeated administration of the LFA-1 signalling mediator over a period of 5 years.
18. The composition for use of embodiment 17, wherein a first administration is followed by repeated administration every 2-7 days.
19. The composition for use according to any one of embodiments 1,3-18, or the LFA-1 signalling mediator for use of embodiments 2-8, 12-14, wherein the cancer is selected from the group consisting of breast cancer, brain cancer, blood forming organ cancer (e.g. Acute Myeloid Leukemia), immune system cancer (e.g. Hodgkin lymphoma), prostate cancer, lung cancer, colon cancer, head and neck cancer, skin cancer, ovary cancer, endometrium cancer, cervix cancer, kidney cancer, lung cancer, stomach cancer, small intestine cancer, liver cancer, pancreas cancer, testis cancer, pituitary gland cancer, blood cancer, spleen cancer, gall bladder cancer, bile duct cancer, esophagus cancer, salivary gland cancer, and the thyroid gland cancer.
20. The composition for use according to any one of embodiments 1,3-19 or the LFA-1 signalling mediator for use of embodiments 2-8, 12-14, or 19 wherein the cancer is a solid tumor and wherein the LFA-1 signalling mediator is administered via intra-tumor injection.

In a first embodiment, the invention relates to a composition for use in cancer immunotherapy comprising an immune system modulator, wherein the immune system modulator enhances the immune response against cancer, and an LFA-1 signalling mediator with moderate LFA-1 stabilization properties, wherein the LFA-1 signalling mediator significantly enhances the anti-cancer immune response.

In another embodiment, the invention relates to an LFA-1 signalling mediator with moderate LFA-1 stabilization properties, wherein the LFA-1 signalling mediator significantly enhances the anti-cancer immune response.

The term “cancer”, as used herein, refers to a disease involving the proliferation of cells whose unique trait—loss of normal controls—results in unregulated growth, lack of differentiation, local tissue invasion, and metastasis. Examples include but are not limited to cancerous diseases and cancerous precursor lesions, including tumorous diseases, including cancer of the breast, brain, blood forming organ (e.g. Acute Myeloid Leukemia), immune system (e.g. Hodgkin lymphoma), prostate, lung, colon, head and neck, skin, ovary, endometrium, cervix, kidney, lung, stomach, small intestine, liver, pancreas, testis, pituitary gland, blood, spleen, gall bladder, bile duct, esophagus, salivary glands, and the thyroid gland.

The term “immunotherapy”, as used herein, refers to the treatment or prevention of a disease, in particular cancer, by a method comprising inducing, enhancing, suppressing or otherwise modifying an immune response.

The term “immune system modulator”, as used herein, refers to agents, drugs, compositions and/or cells that can induce, enhance, suppress or otherwise modify an immune response, in particular as part of an immunotherapy. In some embodiments, the immune system modulator is at least one selected from the group of monoclonal antibodies, modified immune cells, checkpoint inhibitors, small molecules, cytokines, immune adjuvants and IMiDs. In some embodiments, the immune system modulator is at least one selected from the group of monoclonal antibodies, modified immune cells and checkpoint inhibitors. In preferred embodiments of the invention, the immune system modulator is a monoclonal antibody, a modified immune cell or a checkpoint inhibitor. In some embodiments, the monoclonal antibody described herein is selected from the group of naked monoclonal antibody, conjugated monoclonal antibody and bispecific antibody. In some embodiments, the bispecific antibody described herein is a Bi-specific T-cell engager. In some embodiments, the modified immune cell described herein is at least one cell selected from the group of tumor-infiltrating lymphocyte, cell with an engineered T-cell receptor, CAR T-cell and natural killer cells.

The term “immune response”, as used herein, refers to a response by the immune system of a subject. For example, immune responses include, but are not limited to, a detectable alteration (e.g., increase) in Toll receptor activation, lymphokine (e.g., cytokine or chemokine) expression and/or secretion, macrophage activation, dendritic cell activation, T cell activation (e.g., CD4⁺ or CD8⁺ T cells), NK cell activation, and/or B cell activation (e.g., antibody generation and/or secretion). Additional examples of immune responses include binding of an immunogen (e.g., antigen (e.g., immunogenic polypeptide)) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells), and increased processing and presentation of antigen by antigen-presenting cells. An immune response may be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signalling cascade) cell-mediated immune responses (e.g., responses mediated by T cells (e.g., antigen-specific T cells) and non-specific cells of the immune system) and humoral immune responses (e.g., responses mediated by B cells (e.g., via generation and secretion of antibodies into the plasma, lymph, and/or tissue fluids). The term “immune response” is meant to encompass all aspects of the capability of a subject's immune system to respond to antigens (e.g., tumor-associated antigens) and/or immunogens (e.g., both the initial response to an immunogen as well as acquired (e.g., memory) responses that are a result of an adaptive immune response).

The term “LFA-1 signalling mediator”, as used herein, refers to an agent that can induce, enhance, facilitate, suppress or otherwise modify LFA-1 signalling.

The term “LFA-1 stabilization properties”, as used herein, refers to the properties of an LFA1 signalling mediator to reduce the probability of LFA-1 to be in its low-affinity state. An LFA-1 signalling mediator with strong LFA-1 stabilization properties, such as CBR LFA-1/2, is an LFA-1 signalling mediator that induces an LFA-1 mediated lymphocyte adhesion to ICAM-1 substrate with a K_{d}≤10.2 μM in the assay described in “Regulation of integrin affinity on cell surfaces” (Schürpf, Thomas, and Timothy A Springer. The EMBO journal vol. 30,23 4712-27. 23 Sep. 2011). An LFA-1 signalling mediator with moderate LFA-1 stabilization properties according to the invention, is an LFA-1 signalling mediator that induces an LFA-1 mediated lymphocyte adhesion to ICAM-1 substrate with a K_{d}>10.2 μM in the assay described in “Regulation of integrin affinity on cell surfaces” (Schürpf, Thomas, and Timothy A Springer. The EMBO journal vol. 30,23 4712-27. 23 Sep. 2011) and significantly enhances the anti-cancer immune response. In some embodiments, the LFA-1 signalling mediator with moderate LFA-1 stabilization properties according to the invention is an LFA-1 signalling mediator that mediates LFA-1 signalling primarily by enhancing binding to the open headpiece confirmation. Therefore, in these embodiments, the LFA-1 signalling mediator with moderate LFA-1 stabilization properties enhances LFA-1 binding in the open headpiece confirmation more than LFA-1 binding in the assembled CD11a/CD18 heterodimer and the bent LFA-1 confirmation. The binding in the open headpiece confirmation can be determined with flow cytometry using an m24 (M24 clone, Biolegend, category number 363402) (see e.g. FIG. 2i). The binding in the assembled CD11a/CD18 heterodimer can be determined with flow cytometry using a TS2/4 antibody (TS2/4 clone, Biolegend, category number 350602) (see e.g. FIG. 2f). The binding in the in the bent confirmation can be determined with flow cytometry using an HI111 antibody (HI111 clone, Biolegend, category number 301202) (see e.g. FIG. 2g). In some embodiments, the LFA-1 signalling mediator with moderate LFA-1 stabilization properties described herein can enhance the CD3-stimulated m24 and/or KIM127 binding at least about 10%, at least about 20%, at least about 30%, at least about 40%, at least about 50%, at least about 60%, at least about 70%, at least about 80%, at least about 90%, at least about 100%, preferably in an assay as described in FIG. 2h or 2i respectively. In some embodiments, the LFA-1 signalling mediator with moderate LFA-1 stabilization properties described herein can enhance the CD3-stimulated m24 and/or KIM127 binding at least about 10%, at least about 20%, at least about 30%, at least about 40%, at least about 50%, at least about 60%, at least about 70%, at least about 80%, at least about 90%, at least about 100%, more than the CD3-stimulated HI111 and/or TS2/4 binding, preferably as described in the corresponding assays of FIGS. 2f, 2g, 2h, 2i. In some embodiments, the LFA-1 signalling mediator with moderate LFA-1 stabilization properties described herein induces an at least about 10%, at least about 20%, or at least about 30% increase of at least one LFA-1 signalling marker, preferably wherein the LFA-1 signalling marker is CD3/28 mediated % phospho-FAK 397 positivity and/or % TNF positivity, more preferably as detected in the assay described in FIG. 2J or 2K respectively. In some embodiments the LFA-1 signalling mediator with moderate LFA-1 stabilization properties does not or not substantially enhance the binding to the assembled CD11a/CD18 heterodimer and does not or not substantially enhance the bent LFA-1 confirmation. In the context of the invention “not or not substantially enhance LFA-1 binding” means enhancing LFA-1 binding less than 10%, 9%, 8%, 7%, 6%, 5%, 4%, 3%, 2%, 1% or no enhancement. In some embodiment, the thresholds and ranges describing the LFA-1 signalling mediator with moderate LFA-1 stabilization properties are achieved in an assay at a concentration of at least about 0.012 mM, at least about 0.06 mM, at least about 0.12 mM, at least about 0.6 mM, or at least about 1.2 mM. In some embodiment, the thresholds and ranges describing the LFA-1 signalling mediator with moderate LFA-1 stabilization properties are achieved in an assay at least in a concentration range of about 0.012 mM to about 1.2 mM, about 0.06 mM to about 1.2 mM, about 0.12 mM to about 1.2 mM, about 0.6 mM or about 1.2 mM. In some embodiment, the thresholds and ranges describing the LFA-1 signalling mediator with moderate LFA-1 stabilization properties are achieved in an assay at least at the most effective concentration.

Therefore, in some embodiments of the invention, LFA-1 stabilization properties weaker than the LFA-1 stabilization properties CBR LFA-1/2 are considered moderate LFA-1 stabilization properties.

Without being bound to theory, the composition of the invention or the LFA-1 signalling mediator of the invention can enhance the immune response against cancer (FIGS. 1b-f, h,i), by optimizing LFA-1 stabilization. The present inventors have found that a moderate LFA-1 stabilization is surprisingly beneficial for cancer immunotherapy. In contrast, it has been previously suggested that strong LFA-1 stabilization is necessary for LFA-1 affinity on T lymphocytes for ICAM-1 (Schürpf, Thomas, and Timothy A Springer. “Regulation of integrin affinity on cell surfaces.” The EMBO journal vol. 30,23 4712-27. 23 Sep. 2011) or that LFA1 blockade is beneficial for cancer immunotherapy (Cohen S, Haimovich J, Hollander N. Anti-idiotype x anti-LFA-1 bispecific antibodies inhibit metastasis of B cell lymphoma. J Immunol. 2003; 170(5):2695-2701). As such, the invention provided herein is based on the surprising finding that an LFA-1 signalling mediator with moderate LFA-1 stabilization properties rather than an LFA-1 signalling mediator with strong LFA-1 stabilization properties in a composition or alone is able to enhance the immune response during cancer therapy.

In certain embodiments of the invention, the LFA-1 signalling mediator induces selective T-cell mediated killing of cells presenting tumor-associated antigens.

The phrase “selective T-cell mediated killing”, as used herein, refers to a ratio of T-cell mediated killing of cells presenting tumor-associated antigens (e.g. killing of pulsed cells) divided by T-cell mediated killing of cells not presenting tumor-associated antigens (e.g. killing of unpulsed cells) in an assay as described in (Examples, FIG. 4e) being larger than 1.5, preferably larger than 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0.

The term “tumor-associated antigens”, as used herein, refers to a molecule (e.g., a protein or peptide) that is expressed by a tumor-associated cell and either differs qualitatively from its counterpart expressed in normal cells, or is expressed at a higher level in tumor cells than in normal cells. Thus, a tumor-associated antigen can differ from (e.g., by one or more amino acid residues where the molecule is a protein), or it can be identical to its counterpart expressed in normal cells. Some tumor-associated antigens are not expressed by normal cells, or are expressed at a level at least about two-fold higher (e.g., about two-fold, three-fold, fivefold, ten-fold, 20-fold, 40-fold, 100-fold, 500-fold, 1,000-fold, 5,000-fold, or 15,000-fold higher) in a tumor cell than in the tumor cell's normal counterpart.

Any suitable tumor-associated antigen can be used. Tumor-associated antigens include without limitation naturally occurring tumor antigens and modified forms thereof that induce an immune response in a subject, and further include antigens associated with tumor cells and antigens that are specific to tumor cells and modified forms of the foregoing that induce an immune response in a subject. The term tumor-associated antigen further encompasses antigens that correspond to proteins that are correlated with the induction of tumors such as oncogenic virus antigens (e.g., human papilloma virus antigens). Exemplary tumor-associated antigens include, without limitation, HER2/neu and BRCA1 antigens for breast cancer, MART-1/MelanA (melanoma antigen), Fra-1 (breast cancer), NY-BR62, NY-BR85, hTERT, gplOO, tyrosinase, TRP-I, TRP-2, NY-ESO-I, CDK-4, β-catenin, MUM-I, Caspase-8, KIAA0205, SART-I, PRAME, and pi 5 antigens, members of the MAGE family (melanoma antigens), the BAGE family (melanoma antigens), the DAGE/PRAME family (such as DAGE-1), the GAGE family (melanoma antigens), the RAGE family (such as RAGE-I), the SMAGE family, NAG, TAG-72, CA1 25, mutated proto-oncogenes such as p2ras, mutated tumor suppressor genes such as p53, tumor-associated viral antigens (e.g., HPV E6 and E7), the SSX family, HOM-MEL-55, NY-COL-2, HOM-HD-397, HOM-RCC-1.14, HOM-HD-21, HOM-NSCLC-11, HOM-MEL-2.4, HOM-TES-11, RCC-3.1.3, NY-ESO-I, and the SCP family. Members of the MAGE family include, but are not limited to, MAGE-I, MAGE-2, MAGE-3, MAGE-4, MAGE-6, MAGE-11, and MAGE-12. Members of the GAGE family include, but are not limited to, GAGE-I, GAGE-6. See, e.g., the review by Van den Eynde and van der Bruggen, (1997) Curr. Opin. Immunol. 9: 684-693; and Sahin et al., (1997) Curr. Opin. Immunol. 9: 709-716.

The tumor-associated antigen can also be, but is not limited to human epithelial cell mucin (Muc-1; a 20 amino acid core repeat for the Muc-1 glycoprotein, present on breast cancer cells and pancreatic cancer cells), MUC-2, MUC-3, MUC-18, carcino-embryonic antigen (CEA), the raf oncogene product, CA-125, GD2, GD3, GM2, TF, sTn, gp75, EBV-LMP 1 & 2, prostate-specific antigen (PSA), prostate-specific membrane antigen (PSMA), GnT-V intron V sequence (N-acetylglucosaminyltransferase V intron V sequence), Prostate Ca psm, MUM-I-B (melanoma ubiquitous mutated gene product), alpha-fetoprotein (AFP), COI 7-1 A, GA733, gp72, β-HCG, gp43, HSP-70, pi 7 mel, HSP-70, gp43, HMW, HOJ-I, melanoma gangliosides, TAG-72, mutated proto-oncogenes such as p21ras, mutated tumor suppressor genes such as p53, estrogen receptor, milk fat globulin, telomerases, nuclear matrix proteins, prostatic acid phosphatase, protein MZ2-E, polymorphic epithelial mucin (PEM), folate-binding-protein LK26, truncated epidermal growth factor receptor (EGFR), Thomsen-Friedenreich (T) antigen, GM-2 and GD-2 gangliosides, polymorphic epithelial mucin, folate-binding protein LK26, human chorionic gonadotropin (HCG), pancreatic oncofetal antigen, cancer antigens 15-3, 19-9, 549, 195, squamous cell carcinoma antigen (SCCA), ovarian cancer antigen (OCA), pancreas cancer associated antigen (PaA), EBNA (Epstein-Barr Virus nuclear antigen) 1-6, gp75, chimeric protein P210BCR-ABL, lung resistance protein (LRP) Bcl-2, and Ki-67. See, e.g., U.S. Pat. No. 6,537,552; see also U.S. Pat. Nos. 6,815,531; 6,773,707; 6,682,928; and 6,623,739.

The tumor-associated antigen can also be an antibody produced by a B cell tumor (e.g., B cell lymphoma; B cell leukemia; myeloma; hairy cell leukemia), a fragment of such an antibody, which contains an epitope of the idiotype of the antibody, a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR. In one embodiment, the tumor-associated antigen of this invention can be a single-chain antibody (scFv), comprising linked VH, and VL domains, which retains the conformation and specific binding activity of the native idiotype of the antibody.

An LFA-1 signalling mediator with moderate LFA-1 stabilization properties as provided herein can support immune cells in their immune response, e.g., T-cell function (FIG. 2c), cytotoxicity (FIGS. 3d, 3k, 3l, 3o, 4c, 4d), degranulation (FIGS. 3c and 3j) and/or cytokine release (FIGS. 2d and 3p). The T-cell mediated killing of target cells is selective in the presence of an LFA-1 signalling mediator with moderate LFA-1 stabilization properties, while it is non-selective in the presence of an LFA-1 signalling mediator with strong LFA-1 stabilization properties (FIG. 4e).

Accordingly, the invention provided herein is based on the surprising finding that an LFA-1 signalling mediator with moderate LFA-1 stabilization properties induces less unwanted effects, such as killing of non-target cells, than an LFA-1 signalling mediator with strong LFA-1 stabilization properties.

Therefore, in certain embodiments of the invention, the LFA-1 signalling mediator with moderate LFA-1 stabilization properties induces less T-cell mediated killing of cells not presenting tumor-associated antigens than a signalling mediator with strong LFA-1 stabilization properties. The strong LFA-1 stabilization therefore is more likely to induce unwanted side effects by inducing killing of non-target cells.

Accordingly, the invention provided herein is based on the surprising finding that moderate but not strong LFA-1 stabilization properties of an LFA-1 signalling mediator mediate selective killing of target cells.

In certain embodiments of the invention, the LFA-1 signalling mediator with strong LFA-1 stabilization properties is an antibody that binds to the I-EGF-3 binding site of LFA-1, more preferably the LFA-1 signalling mediator with strong LFA-1 stabilization properties is CBR LFA-1/2.

The term “CBR LFA-1/2”, as used herein, refers to a monoclonal antibody as described by Petruzzelli, L et al. (“Activation of lymphocyte function-associated molecule-1 (CD11a/CD18) and Mac-1 (CD11b/CD18) mimicked by an antibody directed against CD18.” Journal of immunology (Baltimore, Md.: 1950) vol. 155,2 (1995): 854-66).

Accordingly, the invention provided herein is based on the surprising finding that an LFA-1 signalling mediator with moderate LFA-1 stabilization properties, but not an LFA-1 signalling mediator with the LFA-1 stabilization properties of CBR LFA-1/2 mediates selective killing of target cells.

In certain embodiments of the invention, the LFA-1 signalling mediator binds to LFA-1, preferably in the extracellular region of LFA-1, more preferably to the β-chain of LFA-1, more preferably to the headpiece of LFA-1, more preferably in the I domain of LFA-1, more preferably to the metal-ion dependent adhesion site of LFA-1 to induce moderate LFA-1 stabilization. Within the present invention, an LFA-1 signalling mediator, particularly an LFA-1 signalling mediator with moderate LFA-1 stabilization properties, can be an antibody, a peptide, a small molecule, or a cation, in particular a divalent cation such as Mg²⁺.

The term “metal-ion dependent adhesion site”, as used herein, refers to a distinct site in the Idomain of the LFA-1 molecule that allows adhesion of metal-ions, such as Mg²⁺ or Mn²⁺.

The term “divalent cation”, as used herein, refers to a positively charged element, atom, or molecule having a valence of plus 2. The term includes metal ions such as Ca²⁺, Zn²⁺, Mn²⁺, Mg²⁺, Fe²⁺, Co²⁺, Ni²⁺ and/or Cu²⁺. In certain embodiments of the invention, divalent cations are salt forms of the ions. Specific examples of divalent salt forms include CaCl2, ZnCl2, MnSO4, MnCl2, and MgCl2 and other combinations of the above exemplary divalent cations in a salt form with, for example, chloride (Cl), sulfate (SO4), acetate (Ac) and/or phosphate (P). Divalent cations and salt forms other than those exemplified above are well known in the art and included in the meaning of the term as it is used herein.

Divalent cations are known to bind to the metal-ion dependent adhesion site of LFA-1 and produce moderate LFA-1 stabilization properties.

Accordingly, the invention provided herein is based on the surprising finding that binding of an LFA-1 signalling mediator to LFA-1, preferably in the extracellular region of LFA-1, more preferably to the β-chain of LFA-1, more preferably to the headpiece of LFA-1, more preferably in the I domain of LFA-1, more preferably to the metal-ion dependent adhesion site of LFA-1 inducing moderate LFA-1 stabilization is able to selectively enhance the immune response of the immune system and/or of an immune system modulator during cancer therapy.

LFA-1 signalling mediators with moderate LFA-1 stabilization properties can be identified by screening for moderate LFA-1 stabilization properties using methods known in the art, for example using a flow-based assay (FIGS. 2f-i, 2l) or by using other methods known to the person skilled in the art such as virtual screening (Shoda M, Harada T, Yano K, et al. Virtual screening leads to the discovery of an effective antagonist of lymphocyte function-associated antigen-1. ChemMedChem. 2007; 2(4):515-521.), V-well adhesion assay (Weetall M, Hugo R, Friedman C, et al. A homogeneous fluorometric assay for measuring cell adhesion to immobilized ligand using V-well microtiter plates. Anal Biochem. 2001; 293(2):277-287.), cell-free ligand-binding assays for integrin LFA-1 (Yuki, Koichi. Methods in molecular biology (Clifton, N.J.) vol. 757 (2012): 73-8), a FRET based quantification and screening technology platform (Chakraborty S, Núñez, Hu S Y, et al. FRET based quantification and screening technology platform for the interactions of leukocyte function-associated antigen-1 (LFA-1) with intercellular adhesion molecule-1 (ICAM-1). PLoS One. 2014; 9(7):e102572. Published Jul. 17, 2014.), confocal on-bead-screening (Hintersteiner, Martin, et al. “Identification and X-ray co-crystal structure of a small-molecule activator of LFA-1-ICAM-1 binding.” Angewandte Chemie International Edition 53.17 (2014): 4322-4326), negative stain electron microscopy (Takagi, J., Petre, B. M., Walz, T., and Springer, T. A. (2002). Global conformational rearrangements in integrin extracellular domains in outside-in and inside-out signaling. Cell 110, 599-611.), crystallography (Xiong, J. P., Stehle, T., Zhang, R., Joachimiak, A., Frech, M., Goodman, S. L., and Arnaout, M. A. (2002). Crystal structure of the extracellular segment of integrin αVβ3 in complex with an Arg-Gly-Asp ligand. Science 296, 151-155.), NMR (Beglova, N., Blacklow, S. C., Takagi, J., and Springer, T. A. (2002). Cysteine-rich module structure reveals a fulcrum for integrin rearrangement upon activation. Nat. Struct. Biol. 9, 282-287), epitope mapping (Lu, C., Ferzly, M., Takagi, J., and Springer, T. A. (2001). Epitope mapping of antibodies to the C-terminal region of the integrin β2 subunit reveals regions that become exposed upon receptor activation. J. Immunol. 166, 5629-5637 and Lu, C., Shimaoka, M., Zang, Q., Takagi, J., and Springer, T. A. (2001).

Locking in alternate conformations of the integrin αLβ2 I domain with disulfide bonds reveals functional relationships among integrin domains. Proc. Natl. Acad. Sci. USA 98, 2393-2398) and/or rapid flow cytometry methods (Crucian, Brian, Mayra Nelman-Gonzalez, and Clarence Sams. “Rapid flow cytometry method for quantitation of LFA-1-adhesive T cells.” Clinical and vaccine immunology 13.3 (2006): 403-408.).

Within the present invention, an LFA-1 signalling mediator with moderate LFA-1 stabilization properties may be selected upon screening using one or more assays, such as, e.g., one of the assays provided above. Accordingly, an LFA-1 signalling mediator as used herein with moderate LFA-1 stabilization properties may be selected on the basis of an assay testing for the effect on the function of the immune system, such as metabolic reprogramming (e.g. FIGS. 2a and 3a), enhanced and selective immune-cell mediated killing (e.g. FIGS. 3d, 3k and 3o) and/or immune synapse formation as described by Somersalo K, et al. (Cytotoxic T lymphocytes form an antigen-independent ring junction. J Clin Invest. 2004; 113(1):49-57) or Franciszkiewicz K, et al (CD103 or LFA-1 engagement at the immune synapse between cytotoxic T cells and tumor cells promotes maturation and regulates T-cell effector functions. Cancer Res. 2013; 73(2):617-628). An LFA-1 signalling mediator with moderate LFA-1 stabilization properties as used herein preferably induces a marked ECAR increase (e.g. FIGS. 2a and 3a), a marked increased killing of tumor cells and/or cells presenting tumor-associated antigens (e.g. FIGS. 3k, 3o), while having selective T-cell mediated killing properties (e.g. FIG. 4e).

A preferred LFA-1 signalling mediator with moderate LFA-1 stabilization properties as used herein may have properties in one or more of the above-mentioned assay(s) that is/are similar to the properties of Mg²⁺ in the one or more assay, preferably similar to the properties of Mg²⁺ in the one or more assay.

In one example, the person skilled in the art identifies an LFA-1 signalling mediator with moderate LFA-1 stabilization properties according to the invention by using two-step screening. In a first step, an assay, e.g., an immune synapse formation assay mentioned above, is used to identify at least one LFA-1 signalling mediator candidate that induces an LFA-1 mediated lymphocyte adhesion to ICAM-1 substrate with a K_{d}>10.2 μM. In a second step, the candidate(s) from the first step is/are identified as LFA-1 signalling mediator(s) with moderate LFA-1 stabilization properties, if the candidate(s) significantly enhance(s) the anticancer immune response. Candidates are preferably selected, if the candidates enhance T-cell mediated killing, more preferably, if the candidates induce selective T-cell mediated killing of cells presenting tumor-associated antigens in an assay described in FIG. 3k in the second step of the screening. The concentration of the LFA-1 signalling mediator in the assay described in FIG. 4e is according to the concentration estimated by the person skilled in the art to be appropriate to mediate LFA-1 signalling. In embodiments of the invention, wherein the LFA-1 signalling mediator is CBR-LFA1/2, the appropriate CBR-LFA1/2 concentration is about 10⁻¹ based on the LFA-1 activity observed at these concentrations in previous studies (Petruzzelli L, Maduzia L, Springer T A. Activation of lymphocyte function-associated molecule-1 (CD11a/CD18) and Mac-1 (CD11b/CD18) mimicked by an antibody directed against CD18. J Immunol. 1995; 155(2):854-866; Grönholm M, Jahan F, Bryushkova E A, et al. LFA-1 integrin antibodies inhibit leukocyte α4β1-mediated adhesion by intracellular signaling. Blood. 2016; 128(9):1270-1281).

Accordingly, the LFA-1 signalling mediator of the invention significantly enhances the anticancer immune response is surprisingly selective and useful for the use in cancer immunotherapy.

Accordingly, the composition of the invention comprising an immune system modulator which enhances the immune response against cancer and an LFA-1 signalling mediator with moderate LFA-1 stabilization properties, wherein the LFA-1 signalling mediator significantly enhances the anti-cancer immune response is surprisingly selective and useful for the use in cancer immunotherapy.

LFA-1 is involved in the process of cell-to-cell contact-mediated killing as well as antibody-mediated killing (Oxford Dictionary of Biochemistry and Molecular Biology. Eds. Cammack, Richard, Teresa Atwood, Peter Campbell, Howard Parish, Anthony Smith, Frank Vella, and John Stirling.: Oxford University Press, 2008).

In certain embodiments of the invention, the immune system modulator is a monoclonal antibody, a modified immune cell or a checkpoint inhibitor (CPI).

The term “antibody”, as used herein, refers to a protein of the immunoglobulin family or a polypeptide comprising fragments of an immunoglobulin that is capable of specifically binding a corresponding antigen. In general, the term “antibody” is used herein in the broadest sense and encompasses various antibody structures, including but not limited to monoclonal antibodies, polyclonal antibodies, multispecific antibodies (e.g., bispecific antibodies), chimeric antibody, fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies or antibodies displayed upon the surface of a phage or displayed upon the surface of a chimeric antigen receptor (CAR) T cell. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265).

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modified “monoclonal” indicates the character of the antibody as being amongst a substantially homogeneous population of antibodies and is not to be construed as requiring production of the antibody by any particular method. As mentioned above, the monoclonal antibodies to be used in accordance with the present invention may be made by the hybridoma method described by Kohler, Nature 256 (1975), 495.

The term “modified” immune cell, as used herein, refers to a cell that is manipulated in vitro in such a way that it imparts an enhanced immune response against cancer after administration of the cell to a subject. In some embodiments of the invention, the modified immune cell originates from a subject and is re-administered to the same subject after manipulation. In some embodiments of the invention, the modified immune cell originates from one subject and is administered to a second subject. Modified immune cells include, but are not limited to, engineered immune cells and/or cells that are activated by an activation protocol and/or expanded by an expansion protocol. The term “engineered” immune cell, as used herein, refers to an immune cell into which an exogenous nucleic acid sequence, such as, for example, a vector, has been introduced and that enhances the immune response against cancer. Engineered immune cells are therefore distinguishable from naturally occurring immune cells that do not contain a recombinantly introduced nucleic acid. Although the immune cells naturally may have receptors for targeting antigens, receiving cytokine signals, and so forth, the immune cells of the present disclosure are non-natural and are engineered directly or indirectly by the hand of man such that they express the desired bipartite or tripartite signalling molecules. The engineered immune cells may be manipulated by recombinant engineering to express one, two, or three of the signalling molecules. Engineering of the cells to express one or more signalling molecules may occur in a single step or in multiple steps, and the manipulation may occur at a single point in time or in successive points in time. In specific aspects, the cells are for adoptive transfer. The cells may be included in a pharmaceutical composition. The cells may be transformed or transfected with one or more vectors as described herein. The recombinant cells may be produced by introducing at least one of the vectors described herein. The presence of the vector in the cell mediates the expression of the appropriate receptor, and in some embodiments one or more constructs are integrated into the genome of the cell. That is, nucleic acid molecules or vectors that are introduced into the host may either integrate into the genome of the host or they may be maintained extrachromosomally. Engineered immune cells include, but are not limited to, CAR T cells, engineered cytotoxic T cells, engineered B cells, engineered granulocytes and/or engineered monocytes, such as engineered macrophages, engineered dendritic cells. In some embodiments, the engineered immune cell described herein is at least one cell selected from the group of CAR T cells, cytotoxic T cells, B cells, granulocytes, NK cells and monocytes.

As used herein, the terms “CPI” or “checkpoint inhibitor” refer to molecules that totally or partially reduce, inhibit, interfere with or modulate one or more checkpoint proteins. Checkpoint proteins regulate T-cell activation or function.

Without being bound to theory, the LFA-1 mediator may enhance cell adhesion, cell migration, and/or cell differentiation via LFA-1 stabilization (Oxford Dictionary of Biochemistry and Molecular Biology. Eds. Cammack, Richard, Teresa Atwood, Peter Campbell, Howard Parish, Anthony Smith, Frank Vella, and John Stirling.: Oxford University Press, 2008; Verma, Navin Kumar, and Dermot Kelleher. “Not just an adhesion molecule: LFA-1 contact tunes the T lymphocyte program.” The Journal of Immunology 199.4 (2017): 1213-1221.). The LFA-1 mediator may be able to enhance the immune response of innate immune cells and/or the immune response of adaptive immune cells. For example, the LFA-1 mediator with moderate LFA-1 stabilization properties may enhance the immune response of T Cells, B Cells, granulocytes and/or monocytes (Oxford Dictionary of Biochemistry and Molecular Biology. Eds. Cammack, Richard, Teresa Atwood, Peter Campbell, Howard Parish, Anthony Smith, Frank Vella, and John Stirling.: Oxford University Press, 2008; Carrasco Y R, Fleire S J, Cameron T, Dustin M L, Batista F D. LFA1/ICAM-1 interaction lowers the threshold of B cell activation by facilitating B cell adhesion and synapse formation. Immunity. 2004; 20(5):589-599). Therefore, the composition of the invention may be particularly useful for the use in cancer immunotherapy, by comprising T Cells, B Cells, granulocytes and monocytes. The immune response of modified immune cells can be particularly enhanced by an LFA-1 mediator (see e.g. FIGS. 3o, 4c). Certain modified immune cells, such as memory T cells, PHA-induced T cell blasts as well REP T cells, have higher LFA-1 expression than naïve CD8⁺ cells (FIGS. 2e and 3e) and are therefore particularly useful for use in the invention. This observation is further supported by the absence of LFA-1 mediator induced metabolic changes (FIG. 2b) and absence of LFA-1 head-piece opening in naïve CD8⁺ cells (FIG. 2l), while cytokine release (FIG. 2d), activation marker (FIG. 2c) and ECAR increase in non-naïve CD8⁺ cells (e.g. EM CD8⁺ cells, PHA Blast cells and REP T) upon LFA-1 mediator action (FIGS. 2a and 2m).

Beside modified immune cells, monoclonal antibodies and/or checkpoint inhibitors are also useful to induce an immune response that can be enhanced by an LFA-1 mediator (FIG. 1h). Checkpoint proteins regulate T-cell activation or function. Particularly central to the immune checkpoint process are the cytotoxic T-lymphocyte-associated antigen 4 (CTLA-4) and programmed death 1 (PD-1) immune checkpoint pathways. The CTLA-4 and PD-1 pathways are thought to operate at different stages of an immune response. CTLA-4 is considered the “leader” of the immune checkpoint inhibitors, as it stops potentially autoreactive T cells at the initial stage of naive T-cell activation, typically in lymph nodes. The PD-1 pathway regulates previously activated T cells at the later stages of an immune response, primarily in peripheral tissues. Progressing patients have been shown to lack of PD-L1 upregulation by either tumor cells or tumor-infiltrating immune cells (Romano E, Romero P. The therapeutic promise of disrupting the PD-1/PD-L1 immune checkpoint in cancer: unleashing the CD8 T cell-mediated antitumor activity results in significant, unprecedented clinical efficacy in various solid tumors. J Immunother Cancer. 2015; 3:15). Immune therapies targeting the PD-L1/PD-1 pathway might thus be especially effective in tumors where this immune-suppressive axis is operational, and reversing the balance towards an immune protective environment would rekindle and strengthen a pre-existing anti-cancer immune response. Monoclonal antibodies can block cellular interactions that negatively regulate T-cell immune responses, such as CD80/CTLA-4 and PD-1/PD-1L, amplifying preexisting immunity and thereby evoking anti-cancer immune responses (Sagiv-Barfi I, Kohrt H E, Czerwinski D K, Ng P P, Chang B Y, Levy R. Therapeutic antitumor immunity by checkpoint blockade is enhanced by ibrutinib, an inhibitor of both BTK and ITK. Proc Natl Acad Sci U S A. 2015; 112(9):E966-E972.). PD-1 thus limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. PD-1 blockade can be accomplished by a variety of mechanisms, including antibodies that bind PD-1 or its ligand, PD-L1. Inhibition of the immune checkpoint pathways has led to the approval of several new drugs: ipilimumab (antiCTLA-4; Yervoy®), pembrolizumab (anti-PD-1; Keytruda®), Cemiplimab(anti-PD-1; Libtayo®), Spartalizumab (anti-PD-1; Novartis®) and nivolumab (anti-PD-1; Opdivo®). Also PD-L1 inhibitors, such as Atezolizumab (MPDL3280), Avelumab (MSB0010718C) and Durvalumab (MEDI4736), tremelimumab (monoclonal antibodies targeting PD-L1) are available. These antagonistic antibodies have been associated with objective clinical responses in cancer patients. Antibodies targeting CTLA-4 are already marketed (e.g. Ipilimumab, Yervoy, Bristol-Myers Squibb, BMS) for metastatic melanoma. Other antibody therapies are anti-PD-L1 (e.g., MPDL3280A, Roche) or anti-PD-1 (e.g., Nivolumab, BMS).

Other immune-checkpoint inhibitors include, without limitation, lymphocyte activation gene3 (LAG-3) inhibitors, such as IMP321, a soluble Ig fusion protein. Other immune-checkpoint inhibitors include B7 inhibitors, such as B7-H3 and B7-H4 inhibitors. In particular, the antiB7-H3 antibody MGA271. Also included are TIM3 (T-cell immunoglobulin domain and mucin domain 3) inhibitors. In certain embodiments, the PD-1 inhibitors include anti-PD-L1 antibodies. In certain other embodiments, the PD-1 inhibitors include anti-PD-1 antibodies and similar binding proteins such as nivolumab (MDX1106, BMS-936558, ONO-4538), a fully human IgG4 antibody that binds to and blocks the activation of PD-1 by its ligands PDL1 and PDL2; CT-011 a humanized antibody that binds PD-1; AMP-224 is a fusion protein of B7-DC; an antibody Fc portion; BMS-936559 (MDX-1105-01) for PD-L1 (B7-H1) blockade. Further examples of PD-L1 inhibitors that can be used in certain embodiments are Atezolizumab (MPDL3280), Durvalumab (MEDI4736) and Avelumab (MSB0010718C). The preferred checkpoint inhibitors of the present invention are thus those for PD-1 and PD-L1. In certain embodiments of the invention, the PD-1/PD-L1 inhibitor is an inhibitor selected from the group consisting of nivolumab, pembrolizumab, cemiplimab, spartalizumab, atezolizumab, durvalumab and avelumab. The LFA-1 mediator can enhance the anti-cancer immune response of antibodies against the MC38-OVA (FIG. 1b) and/or the immune response of anti-PD1 antibody (FIGS. 1h and 1i). Accordingly, the composition of the invention comprising a monoclonal antibody, a modified immune cell and/or a checkpoint inhibitor which enhances the immune response against cancer and an LFA-1 signalling mediator with moderate LFA-1 stabilization properties, wherein the LFA-1 signalling mediator significantly enhances the anti-cancer immune response is surprisingly selective and useful for the use in cancer immunotherapy.

A high concentration of the LFA-1 signalling mediator of the invention, of the composition of the invention or of one or more ingredients of the composition of the invention at the target site may be beneficial for the anti-cancer immune response, while a high concentration of the LFA-1 signalling mediator of the invention, of the composition of the invention or of one or more ingredients of the composition of the invention at the non-target site may induce unwanted effects. A high concentration of the LFA-1 signalling mediator of the invention, of the composition of the invention or of one or more ingredients of the composition of the invention at the target site may be achieved by route of administration and/or by support of a carrier.

The LFA-1 signalling mediator of the invention, the composition of the invention or one or more ingredients of the composition of the invention (and any additional therapeutic agent) can be administered by any suitable means, including parenteral, intrapulmonary, and intranasal, and, if desired, e.g., for local treatment, intra-tumoral, intralesional, intrathecally, intrauterine or intravesical administration. Parenteral infusions include subcutaneous, intramuscular, intravenous, intraarterial, intraperitoneal, or subcutaneous administration. In certain embodiments of the invention, the LFA-1 signalling mediator of the invention, the composition of the invention, or one or more ingredients of the composition of the invention may have appropriate properties to be administered (and absorbed) intradermal, intravaginally, orally, topically, inhalationally, intranasally, transdermally, rectally to act locally and/or systemically. Administration techniques that can be employed with the agents and methods described herein are found in, e.g., Goodman and Gilman, The Pharmacological Basis of Therapeutics, current ed.; Pergamon; and Remington's, Pharmaceutical Sciences (current edition), Mack Publishing Co., Easton, Pa., which are incorporated herein by reference.

In certain embodiments, the invention relates to the use of the LFA-1 signalling mediator of the invention or the composition of the invention for use in cancer immunotherapy of a solid tumor, wherein the LFA-1 signalling mediator is administered via intra-tumor injection.

The term “solid tumor”, as used herein, refers to a tumor that forms a discrete tumor mass. Examples of solid tumors within the scope of this method include colon, rectum, kidney, bladder, prostate, brain, breast, liver, lung, skin (e.g., melanoma) and head and neck tumors.

The terms “administer”, “administering”, “administration”, and the like, as used herein, refer to methods that may be used to enable the delivery of compositions to the desired site of biological action.

The term “intra-tumor injection”, as used herein, refers to mechanical device-mediated administration into a tumor, into a tumor environment and/or into the tissue containing one or more tumors.

By intra-tumor injection, a high local concentration of the LFA-1 signalling mediator of the invention or of the composition of the invention can be reached in the tumor environment without drastically increasing systemic concentration. Certain LFA-1 signalling mediators, such as Mg²⁺, get cleared quickly from the desired site of biological action or may exhibit unwanted effects (e.g., at non-target sites) at the concentrations most beneficial to enhance the immune response at the target site. Intra-tumor injections of the LFA-1 signalling mediator have proven to be surprisingly useful, to maintain the LFA-1 signalling mediator in a therapeutic range at the target site (FIGS. 1c, d, h and i).

In certain embodiments of the invention, the LFA-1 signalling mediator of the invention the composition of the invention additionally comprises a carrier for targeted delivery of the LFA-1 signalling mediator.

The term “carrier”, as used herein, refers to any pharmaceutically acceptable solvent, suspending agent, vehicle agent, drug, composition, device, tool, or combination thereof that allows targeted delivery.

The term “targeted delivery”, as used herein, refers to a certain way of delivery that allows increasing a concentration and/or an effect of an active agent more in at least one target site than in at least one non-target site.

The use of a carrier may increase local action of the LFA-1 signalling mediator of the invention or of the composition of the invention at the target site, e.g., in the tumor environment. In certain embodiments of the invention, the carrier achieves targeted delivery of the LFA-1 signalling mediator or of at least one ingredient of the composition of the invention by delaying the release of the LFA-1 signalling mediator or the ingredient(s) before arrival at the target site (e.g., by liposome encapsulation), by reducing clearance and/or metabolization of the ingredient(s) at the target site and/or by limiting the effect of an interfering agent at the target site (e.g., calcium chelator).

The targeted delivery and/or delayed release of the LFA-1 signalling mediator of the invention or of the ingredient(s) of the composition of the invention for use in cancer immunotherapy before arrival at the target site may be achieved by any method known by the person skilled in the art. In certain embodiments of the invention, the carrier achieves targeted delivery and/or delayed-release via a plurality of membrane-forming molecules.

The term “membrane-forming molecule”, as used herein, refers to a molecule that allows the formation of a biological membrane or is able to integrate into a biological membrane. The membrane may form a mono-, bilayer sheet or a capsule, such as a liposome or a micelle. In certain embodiments, the capsules contain at least one ingredient of the composition for use in cancer and may further carry medical agents, diagnostic agents, nutritional agent, a radiation sensitizer, a contrast agent, an enzyme, nucleic acid, an antibody, a growth factor, a protein, a peptide, a carbohydrate, a targeting group or combinations of those.

In certain embodiments of the invention, the membrane-forming molecule is a capsule forming lipid. In particular, the delayed release may be achieved by a carrier binding and/or encapsulating the LFA-1 signalling mediator or at least one of the ingredients of the composition of the invention. In certain embodiments of the invention, the carrier comprises polymerizable lipid amphiphiles to generate crosslinked liposomes with higher stability (O'Brien et al., 1998, Acc. Chem. Res. 31:861-868; Moon, J. J., Yuchen, F. A. N., Sandev, P., & Bazzill, J. (2019). U.S. Pat. No. 10,307,491. Washington, DC: U.S. Patent and Trademark Office). Examples include, but are not limited to, DOTAP, DOPE, DOBAQ, or DOPC. In some embodiments of the invention, the carrier comprises a functionalized lipid (e.g., with maleimide or dibenzocyclooctyne (DBCO)).

In certain embodiments of the invention, immune cell-linked (e.g. T-cell-linked) synthetic nanoparticles are used as a carrier to the target site (e.g., into the immunological synapse), for therapeutically modulating immune signalling events. In certain embodiments the carrier forms covalent coupling of maleimide-functionalized nanoparticles to free thiol groups on T cell membrane proteins for delivery of the LFA-1 signalling mediator or at least one of the ingredients of the composition of the invention to the T-cell synapse. The carrier may support delivery of the LFA-1 signalling mediator or at least one of the ingredients of the composition of the invention as described by Stephan, Matthias T., et al. (“Synapse-directed delivery of immunomodulators using T-cell-conjugated nanoparticles.” Biomaterials 33.23 (2012): 57765787).

In some embodiments of the invention, the carrier forms stimuli-responsive liposomes for drug delivery as described in “Stimuli-responsive liposomes for drug delivery” (Lee, Y., and D. H. Thompson. Wiley Interdisciplinary Reviews: Nanomedicine and Nanobiotechnology 9.5 (2017): e1450.).

In some embodiments of the invention, the carrier is a carrier-antibody that is conjugated to the LFA-1 signalling mediator of the invention or at least one of the ingredient(s) of the composition of the invention. The antibody may bind in the target site (e.g., in the tumor environment), or on the tumor cells in order to deliver the LFA-1 signalling mediator of the invention or at least one of the ingredient(s) of the composition of the invention to the target region. In some embodiments of the invention, the carrier-antibody detaches from the LFA-1 signalling mediator of the invention or at least one of the ingredient(s) of the composition of the invention upon binding in the target region. In some embodiments of the invention, the immune system modulator also fulfils the function of a carrier for the LFA-1 signalling mediator.

Reducing clearance and/or metabolization of the LFA-1 signalling mediator of the invention or of at least one ingredient of the composition for use in cancer immunotherapy may be achieved by drug-induced alteration of the metabolism. In an example, clearance of the LFA1 signalling mediator (e.g., Magnesium) is reduced by a parathyroid extract (Gill Jr, JOHN R., NORMAN H. Bell, and FREDERIC C. Bartter. “Effect of parathyroid extract on magnesium excretion in man.” Journal of applied physiology 22.1 (1967): 136-138). In some embodiments of the invention, the carrier is a mechanical device to increase the concentration of the LFA-1 signalling mediator of the invention or of at least one of the ingredient(s) of the composition for use in cancer immunotherapy. In an example, the carrier is a device for increasing plasma concentration of the LFA-1 signalling mediator (e.g. Magnesium) by hemolysis.

Limiting the effect of an interfering agent at the target site to enhance the action of the LFA-1 signalling mediator of the invention or of at least one ingredient of the composition for use in cancer immunotherapy may be achieved, e.g., by using chelates. In one example, the carrier comprises the calcium chelator EGTA to enhance the action of the LFA-1 signalling mediator (Lomakina, Elena B., and Richard E. Waugh. “Micromechanical tests of adhesion dynamics between neutrophils and immobilized ICAM-1.” Biophysical journal 86.2 (2004): 12231233).

Accordingly, the invention provided herein is based on the finding that locally increased concentration (e.g., by intra-tumor injection or by a carrier) of the LFA-1 signalling mediator of the invention or at least one ingredient of the composition of the invention may impart a surprisingly enhanced the immune response during cancer therapy.

Dosing can be by any suitable route, e.g., by injections, such as intravenous, subcutaneous, or intra-tumoral injections, depending in part on whether the administration is brief or chronic. Various dosing schedules, including but not limited to single or multiple administrations over various time-points, bolus administration, and pulse infusion are contemplated herein.

The LFA-1 signalling mediator of the invention, the composition of the invention or ingredients of the composition of the invention would be formulated, dosed, and administered in a fashion consistent with good medical practice. Factors for consideration in this context include the particular type of cancer being treated, the particular subject being treated, the clinical condition of the subject, the progression of cancer, the site of delivery of the agent(s), the method of administration, the scheduling of administration, and other factors known to medical practitioners.

The effective amount of the carrier depends on the amount of the LFA-1 signalling mediator of the invention, the composition of the invention or the amount at least one ingredient of the composition of the invention presents in the formulation, the type and progression of cancer or treatment, and other factors.

In embodiments of the invention, wherein the carrier(s) is/are directly bound to the LFA-1 signalling mediator of the invention or at least one ingredient of the composition of the invention, the carrier(s) is/are generally used in the same dosage ranges and with administration routes as described herein, or about from 1 to 99% of the dosages described herein, or in any dosage and by any route that is empirically/clinically determined to be appropriate.

In embodiments of the invention, wherein the carrier(s) form(s) a membrane, the amount of the carrier may be higher than the amount of the LFA-1 signalling mediator of the invention or at least one other ingredient of the composition of the invention, such as 2 times, 3 times, 5 times, 10 times, 50 times, 100 times, or more than 100 times higher than the amount of the LFA-1 signalling mediator of the invention or at least one other ingredient of the composition of the invention administered at the same time, depending on the factors mentioned above.

In certain embodiments of the invention, the immune system modulator is an antibody or a checkpoint inhibitor, and the dose is about 1 μg/kg to 15 mg/kg (e.g. 0.1 mg/kg-10 mg/kg), depending on the factors mentioned above.

In certain embodiments of the invention, the immune system modulator is a modified immune cell, and the total dose of the immune system modulator for one therapy cycle is typically about 1×10⁴/kg to 1×10¹⁰/kg modified immune cells or more, depending on the factors mentioned above.

In certain embodiments of the invention, the immune system modulator acts as a carrier for the LFA-1 signalling mediator with moderate LFA-1 stabilization properties and the immune system modulator is dosed in a similar dosage range, preferably in about an equimolar dosage range as the LFA-1 signalling mediator with moderate LFA-1 stabilization properties.

In certain embodiments of the invention, the LFA-1 signalling mediator with moderate LFA-1 stabilization properties is a divalent cation and is administered in a solution with a concentration in the range of 0.5-15 mM, preferably 0.9-10 mM, more preferably 1.5-5 mM, in particular about 3 mM (FIGS. 1b, c d, h and i).

In certain embodiments of the invention, the LFA-1 signalling mediator with moderate LFA-1 stabilization properties is an antibody, and the dose is about 1 μg/kg to 15 mg/kg (e.g., 0.1 mg/kg-10 mg/kg), depending on the factors mentioned above.

In certain embodiments of the invention, the LFA-1 signalling mediator with moderate LFA-1 stabilization properties is a peptide, and the dose is about 1 μg/kg to 15 mg/kg (e.g., 0.1 mg/kg-10 mg/kg), depending on the factors mentioned above.

In certain embodiments of the invention, the LFA-1 signalling mediator with moderate LFA-1 stabilization properties is a small molecule, and the dose is about 1 μg/kg to 15 mg/kg (e.g., 0.1 mg/kg-10 mg/kg), depending on the factors mentioned above.

The dose of the LFA-1 signalling mediator of the invention, the composition of the invention or at least one ingredient of the composition of the invention can be an initial candidate dosage for administration to the patient, whether, for example, by one or more separate administrations, or by continuous infusion.

In some embodiments of the invention, the immune system modulator and the LFA-1 signalling mediator are administered simultaneously or sequentially.

The term “simultaneously”, as used herein, refers to the administration of more than one drug at the same time, but not necessarily via the same route of administration or in the form of one combined formulation. For example, one ingredient of the composition of the invention may be provided orally whereas another ingredient of the composition of the invention may be provided intravenously during a patient's visit to a hospital.

In certain embodiments of the invention, the composition of the invention or ingredients of the composition of the invention is/are suitably administered to the patient at one time.

The term “sequentially”, as used herein, refers to the administration of a first ingredient of the composition of the invention if followed, immediately or in time, by the administration of a second ingredient of the composition of the invention.

In certain embodiments of the invention, the immune system modulator or the LFA-1 signalling mediator may have an effect (e.g., priming and/or activation) on the immune system. Depending on factors for relevant scheduling of administration, such as the onset and duration of this effect on the immune system, it is beneficial to administer the immune system modulator and the LFA-1 signalling mediator simultaneously or sequentially.

The immune system modulator and the LFA-1 signalling mediator may differ more factors for scheduling of administration, such as in pharmacokinetic and pharmacodynamic properties and/or in influencing the pharmacokinetic and pharmacodynamic properties of the respective other.

Further factors for scheduling of administration include the particular type of cancer being treated, the particular subject being treated, the clinical condition of the subject, the progression of cancer, the site of delivery of the agent(s), the method of administration, and other factors known to medical practitioners.

In certain embodiments of the invention, the immune system modulator (e.g., modified T cells) is preincubated in a medium containing the LFA-1 signalling mediator (e.g., Mg²⁺) in high concentration, before simultaneous administration to a subject, in order to avoid toxic effect of high LFA-1 signalling mediator concentrations.

In certain embodiments of the invention, the immune system modulator and the LFA-1 signalling mediator with moderate LFA-1 stabilization properties are simultaneously administrated to enable the immune system modulator to acts as a carrier for the LFA-1 signalling mediator with moderate LFA-1 stabilization properties.

In certain embodiments of the invention, the immune system modulator and the LFA-1 signalling mediator with moderate LFA-1 stabilization properties are simultaneously administrated to enable a carrier to support the targeted delivery of several ingredients of the composition of the invention.

In some embodiments of the invention, the immune system modulator and the LFA-1 signalling mediator are administered sequentially.

In certain embodiments of the invention, immune system modulator and the LFA-1 signalling mediator are administered sequentially with a time difference of 1, 5, 10, 15, 20, 30, 45 minute(s), 1, 2, 3, 4, 6, 8, 12, 16 hour(s), 1, 1.5, 2, 2.5 3, 4, 5, 7, 10, 12, 14, 16, 24 days (e.g., FIGS. 1b, c, d, e, h and i)), depending in part on the factors for scheduling of administration mentioned above.

In some embodiments of the invention, the immune system modulator is administered first, followed by the repeated administration of the LFA-1 signalling mediator over a period of less than 5 years.

In certain embodiments of the invention, the LFA-1 signalling mediator of the invention, the composition of the invention and/or at least one ingredient of the composition of the invention is/are suitably administered to the patient over a series of treatments and/or treatment cycles. Typically, the LFA-1 signalling mediator is administered over a period of weeks to months. In cases, where the LFA-1 signalling mediator shows one or more desired effects and is acceptably tolerated the LFA-1 signalling mediator can also be administered over years.

In certain embodiments of the invention, the LFA-1 signalling mediator (e.g., Mg²⁺) is administered repeatedly in order to maintain the LFA-1 signalling mediator concentration in a subject (FIGS. 1a and 1h). This repeated administration may be of particular benefit, in embodiments of the invention wherein the half-life of the LFA-1 signalling mediator has a shorter than the half-life than the immune system modulator. In such an embodiment of the invention, the LFA-1 signalling mediator may be administered repeatedly (e.g every 0.5, 1, 1.5, 2, 2.5 or 3 day(s)) over a period of, e.g., 1 week, 2 weeks, 3 weeks or 4 weeks, depending in part on the factors for scheduling of administration mentioned above.

In other embodiments, the effect of the immune system modulator (e.g., application of modified immune cells) is not primarily dependent on half-life and may exhibit a prolonged effect that can be enhanced by the LFA-1 signalling mediator of the invention. In these embodiments, the period of repeated administration of the LFA-1 signalling mediator may also be longer, such as 5 weeks, 6 weeks, 2 months or longer, depending in part on the factors for scheduling of administration mentioned above.

In some embodiments of the invention, a first administration is followed by repeated administration every 2-7 days.

To maintain a high concentration of the composition of the invention at the target site the composition of the invention is repeatedly administered at least every 7 days, preferably every 6 days, preferably every 5 days, preferably every 4 days, preferably every 3 days, preferably every 2.5 days, preferably every 2 days (FIGS. 1a and 1g).

For repeated administrations over several days or longer, depending on the type of cancer and target site, the treatment would generally be sustained until a desired suppression of disease symptoms occurs. One exemplary dosage of the composition of the invention or an ingredient of the composition of the invention would be in the range from about 0.05 mg/kg to about 10 mg/kg. Thus, one or more doses of about 0.5 mg/kg, 2.0 mg/kg, 4.0 mg/kg, or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses, may be administered. However, other dosage regimens may be useful. The progress of this therapy is easily monitored by conventional techniques and assays.

In certain embodiments of the invention, the immune system modulator is a modified immune cell, and the patient's tolerance is investigated, by injection the total number of cells over several courses, such as three courses according to a pattern of 10% on the first day, 30% on the second day, and 60% on the third day.

Accordingly, the invention provided herein is based on the finding that preferred administration patterns of the composition of the invention are able to surprisingly enhance the immune response of an immune system modulator during cancer therapy.

In some embodiments of the invention, the cancer is selected from the group consisting of cancers of the breast, brain, blood forming organ (e.g. Acute Myeloid Leukemia), immune system (e.g. Hodgkin lymphoma), prostate, lung, colon, head and neck, skin, ovary, endometrium, cervix, kidney, lung, stomach, small intestine, liver, pancreas, testis, pituitary gland, blood, spleen, gall bladder, bile duct, esophagus, salivary glands, and the thyroid gland.

The LFA-1 signalling mediator of the invention and the composition of the invention are particularly useful for cancers in organs or tissues that are accessible for immune cells.

Accordingly, the invention provided herein is based on the finding that the composition of the invention is surprisingly useful for use in immunotherapy of a cancer selected from the group consisting of breast cancer, brain cancer, blood forming organ cancer (e.g. Acute Myeloid Leukemia), cancer of the immune system (e.g. Hodgkin lymphoma), prostate cancer, lung cancer, colon cancer, head and neck cancer, skin cancer, ovary cancer, endometrium cancer, cervix cancer, kidney cancer, lung cancer, stomach cancer, small intestine cancer, liver cancer, pancreas cancer, testis cancer, pituitary gland cancer, blood cancer, spleen cancer, gall bladder cancer, bile duct cancer, esophagus cancer, salivary glands cancer, and/or the thyroid gland cancer.

In some embodiments of the invention, the cancer is selected from the group consisting of cancers of the immune system, thymus, spleen, bone marrow.

The LFA-1 signalling mediator of the invention and the composition of the invention are particularly useful for use in treating cancers that are accessible for T cells.

Accordingly, the invention provided herein is based on the finding that the LFA-1 signalling mediator of the invention and the composition of the invention are surprisingly useful for use in immunotherapy of a cancer selected from the group consisting of cancers melanoma, lung cancer, kidney cancer, bladder cancer, head and neck cancer, Hodgkin lymphoma, bladder cancer, Merkel-cell carcinoma, and/or urothelial carcinoma.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated.

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

### BRIEF DESCRIPTION OF FIGURES

FIG. 1: Intratumoral magnesium administration improves adaptive anti-tumor immunity. (a) Schematic of experimental design. B1/6 mice, either immunized with OVA or left untreated, were inoculated subcutaneously with MC38-OVA tumor cells bilaterally on the flanks. From day 7, injection of 3 mM NaCl solution was applied in left flank tumor, whereas 3 mM MgCl₂ solution was injected in contralateral tumor. This regimen was repeated every third day for a total of 8 cycles. (b) Tumor growth curves in non-immunized mice (n=20) (left panel), and immunized mice (n=19) (right panel). (c) Tumor growth curves in ±CD8 depleted mice (n=6-17). Results were pooled from 2 independent experiments, with n=6-12 mice each. (d) Absolute numbers of tumor-infiltrating CD8⁺ T cells. (e) Number of tumor-infiltrating CD8⁺ T cells positive for Ki67 (left panel), Granzyme B (middle panel) and CD25 (right panel). (f) Cell number of tumor-infiltrating CD8⁺ T cells expressing PD-1 and Tim3. (g) Schematic of experimental design. B1/6 mice, immunized with OVA, were inoculated subcutaneously with MC38-OVA tumor cells unilaterally on the flank. From day 5, intratumoral injections of either 3 mM NaCl or 3 mM MgCl₂ were initiated, and repeated every third day for 8 cycles. Mice were additionally injected with 200 μg i.p. of isotype control (IgG2a) or anti-PD-1 Ab on day 9, 12, and 15. (h) Tumor growth curves (n=13-14) and (i) host survival (n=13-14). Results were pooled from 2 independent experiments (b, h, i).

Data are presented as mean±SEM (B,C,H), median±IQR with each symbol representing one mouse (D,E,F) and statistical significance was assessed by two-way analysis of variance (ANOVA) with Bonferroni correction (B,C and H), unpaired two-tailed Student's t test (D,E), two-way ANOVA with Sidak corrected multiple comparison test (F) and log-rank Mantel-Cox test (I). *p<0.05, **p<0.01, ***p<0.001, ****p<0.0001. i.p. intraperitoneal; Ab Antibody

FIG. 2: Extracellular magnesium promotes memory-specific activation via LFA-1. Glycolytic switch of human EM CD8 T cells (a) and naïve CD8 T cells (b) subsets upon injection of anti-CD3 Ab only, or anti-CD3 and anti-CD28 Ab, was assessed by metabolic flux analysis in medium containing either 1.2 mM Mg²⁺, 0 mM Mg²⁺, or medium which was reconstituted from 0 mM to 1.2 mM Mg²⁺ immediately prior to activation (0→1.2 mM Mg²⁺). (a) Results for human effector memory (EM) and (b) for human naïve CD8 T cells. Glycolytic switch was quantified by subtracting maximal ECAR from baseline ECAR measurements. (c) Flow cytometric analysis of surface activation markers on human EM CD8 T cells 24 hours after activation by plate-bound anti-CD3 Ab and soluble anti-CD28 Ab, in 1.2 mM and 0 mM Mg²⁺ media, respectively. (d) Abundance of inflammatory cytokines in corresponding EM CD8 T cell culture supernatants, determined by CBA. (e) CD11a (LFA-1) surface expression on human naïve and EM CD8 T cells as well PHA T cell blasts. Flow cytometry-based assessment of total LFA-1 expression assessed by mAb TS2/4 (f), closed conformation of αL by mAb HI111 (g), extended conformation of LFA-1 by mAb Kim127 (h), open head-piece conformation of LFA-1 by mAb m24 (i), phosphorylation of focal adhesion kinase (FAK) (h) as well TNF expression (k)±TCR-induced activation in different extracellular Mg²⁺ concentrations. (l) Flow cytometry-based assessment of LFA-1 with open-head piece conformation on human EM and naïve CD8 T cells using the open head-piece reporter mAb m24. (m) Assessment of glycolytic switch of EM CD8 T cells. (n) Assessment of glycolytic switch of naïve CD8 T cells. Assessment of TCR stimulation-induced LFA-1 activation with open head-piece conformation (o) as well of glycolytic switch (p) and degranulation (q) on human PHA T cell blasts±treatment with BIRT377 (50 μM).

Each symbol represents an individual healthy human donor, graphs show pooled results from 2-4 independent experiments, bars indicate mean±SD (a-c, e-q) and bars indicate median±interquartile range (d). Statistical significance was assessed by repeated-measures one-way ANOVA with Sidak's multiple comparison test (a, b, e, l-q), unpaired two-tailed Student's with Holm-Sidak corrected multiple comparisons (c), Mann-Whitney test (d). *p<0.05, **p<0.01, ***p<0.001, ****p<0.0001. mAb monoclonal antibody; Mg²⁺ Magnesium; TNF Tumor necrosis factor; gMFI geometric mean fluorescent intensity

FIG. 3: Magnesium regulates LFA-1 mediated T cell activation and cytotoxicity as well BiTE- and CAR T-cell functionality. (a) Glycolytic switch of murine WT and LFA-1^{-/-} CTLs upon injection of anti-CD3/28 Ab in medium containing 1.2 mM Mg²⁺ and 0 mM Mg²⁺, presented as quantification of summarized data with n=4 mice. (b) Calcium flux in WT and LFA-1^{-/-} Jurkat T cells stimulated with anti-CD3 Ab in 1.2 mM and 0 mM Mg²⁺ medium, respectively. Cells were loaded with Fluo4, and signal intensity as recorded by plate reader, was normalized to unstimulated baseline values. Data is presented as quantification of area under the curve of 3 independent experiments in duplicates (c) Expression of degranulation marker CD107a on murine WT and LFA-1^{-/-} CTLs, after 8 hours of incubation with plate-bound anti-CD3 Ab and soluble anti-CD28 Ab, in medium containing 1.2 mM Mg²⁺, 0 mM Mg²⁺, or 1.2 mM Mg 2+±BIRT377 (50 μM). Quantified results of n=3 mice with 3 technical replicates each. (d) Caspase-3 activity in EL4 target cells after co-culture with murine WT and LFA-1^{-/-} CTLs under the conditions as in (c) for 4 hours, in presence of 10 μg/ml PHA. The effector to target cell ratio was 3:1. Results are quantified of n=3 mice with 3 technical replicates each. (e) Representative CD11a (LFA-1) surface expression on human naïve and EM CD8 T cells, PHA T cell blasts and REP T cells, assessed by flow cytometry. Flow cytometry-based assessment of inactivate LFA-1 with bent conformation (f), extended conformation with β2 leg extension (g), LFA-1 with open head-piece (h), phosphorylation of FAK, expression of degranulation marker CD107a (j) on REP T cells after co-incubation with T2 target cells (pulsed with 10⁻⁸ M 9c peptide) in same condition as in (c). The effector to target cell ratio was 1:1. Data is presented as quantified results of n=5 healthy donors (f, g, h), n=3 healthy donors with 2 technical replicates each or n=1 healthy donor with 4 technical replicates (j). (k) Caspase-3 activity in 9c peptide pulsed (10⁻⁸ M) T2 target cells, 45 min after co-culturing with REP T cells using conditions as in (c). The effector to target cell ratio was 1:1. Data is presented as quantified results of n=1 healthy donor with 4 technical replicates. (l) Box plots representing flow cytometric assessment of caspase-3 activity in Ramos target cells after 3.5 h of co-culture with PHA-blasts of n=5 healthy donors at Mg²⁺ and Blinatumomab concentrations as indicated. (m) Flow cytometry-based assessment of activation-induced LFA-1 head-piece opening on PHA-blasts after 30 min of co-culture with Ramos cells at a Blinatumomab concentration of 300 pg ml⁻¹, n=5 healthy donors. (n) Representative histogram of CD11a expression on PHA-blasts, untransduced T cells as well anti-CD19 expressing CAR T cells. (o) Cytotoxicity assay with anti-PSMA CAR T cells and UTD T cells co-cultured with PSMA+ PC3-PIP cell line, in medium containing 0.6 mM or 0 mM Mg²⁺. Cytotoxicity is reported as total area under the curve of the fluorescence driven by incorporation of cytotoxic green reagent in dead target cells (green area per μm²). The effector to target cell ratio was 2:1. Pooled results of n=6 from 2 independent experiments are shown. (p) Abundance of IFNγ in cell culture supernatants corresponding to the conditions depicted in (o) at 24 hours was determined by ELISA (n=8, 3 independent experiments). (q) Tumor growth curves of mice placed on either Mg²⁺-depleted diet or respective control diet and treated with either anti-PSMA CAR T cells, untransduced T cells or saline solution. Representative of n=2 independent experiments with n=6 mice per group.

Data are presented as mean±SD(a-d, f-k, m,p), ±IQR (l), ±SEM (o, q). Statistical significance was assessed by ordinary one-way ANOVA with Sidak's multiple comparison test (a-d, i-k, l), RM one-way ANOVA with Sidak's multiple comparison (f, g, h), two-way ANOVA with post hoc Tukey test (o, q) and unpaired two-tailed Student's t-test (m, p). Ab antibody; EM effectormemory; Mg²⁺ Magnesium; CTL Cytotoxic T lymphocyte; PSMA prostate-specific membrane antigen; CAR chimeric antigen receptor; UTD untransduced; PHA Phytohaemagglutinin, REP T cells rapid expansion protocol T cells.

FIG. 4: Magnesium regulates LFA-1 mediated T cell activation and cytotoxicity within physiologic range
(a) Metabolic flux analysis of human EM CD8 T cells upon supra-physiologic activation with anti-CD3 and anti-CD28 Ab, and additional injection of secondary, cross-linking anti-CD3/28 Ab, in medium containing 1.2 mM Mg²⁺, 0 mM Mg²⁺, or medium which was reconstituted to 1.2 mM Mg²⁺ immediately prior to activation (0→1.2 mM Mg²⁺). Pooled results from n=6 healthy donors from 3 independent experiments. (b) CD69 expression of PHA T cell blasts upon activation with indicated concentrations of plate-bound anti-CD3 mAb in ±Mg²⁺ containing medium. (c) Caspase-3 activity in EL4 target cells after co-culture for 4 hours with murine WT and LFA-1^{-/-} CTLs in medium containing 1.2 mM Mg²⁺ or 0 mM Mg²⁺, in presence of PHA at the indicated concentrations. The effector to target cell ratio was 3:1. Pooled results, n=3-4, from 2 independent experiments. (d) Cytotoxicity assay using WT and LFA-1^{-/-} OT1 CTLs, co-cultured for 4 hours in medium containing 1.2 mM Mg²⁺ or 0 mM Mg²⁺ with luciferase-expressing EL4 target cells (pulsed with altered OVA peptides at 10^−6 M). Cytotoxicity was quantified after adding luciferin to medium and measuring luminescent signal intensity. The effector to target cell ratio was 3:1 (n=4 mice). (e) Cytotoxicity of human REP T cells co-cultured for 4 hours with luciferase expressing T2 target cells (±pulsed with 9c peptide at 10⁻⁸ M) in medium containing activating anti-LFA-1 Ab CBR-LFA1/2 (10 μg/ml) or isotype control Ab (10 μg/ml), and 1.2 mM or 0 mM Mg²⁺. Cytotoxicity was quantified after adding luciferin to medium and measuring luminescent signal intensity. The effector to target cell ratio was 2:1. Representative experiment with n=1 healthy donor and 3-6 technical replicates.

Data are presented as mean±SD. Statistical significance was assessed by ordinary one-way ANOVA with Sidak's multiple comparison test (a, d, e)

### DETAILED DESCRIPTION OF THE INVENTION

As used herein, the articles “a” and “an” refer to one or to more than one, i.e., to at least one, of the grammatical object of the article. By way of example, “an element” means one element or more than one element. The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of ±20% or ±10%, in some instances ±5%, in some instances ±1%, and in some instances ±0.1% from the specified value, as such variations are appropriate to perform the disclosed methods. The term “treatment” (and grammatical variations thereof such as “treat” or “treating”), as used herein, refers to at least one intervention in an attempt to alter the natural course of the individual being treated, and can be performed either for prophylaxis or during the course of at least one pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. Treatment includes any beneficial or desirable effect on the symptoms or pathology of a disease or pathological condition and may include even minimal reductions in one or more measurable markers of the disease or condition being treated, e.g., cancer. “Treatment” does not necessarily indicate complete eradication or cure of the disease or condition, or associated symptoms thereof. The terms “patient”, “subject”, “individual”, and the like are used interchangeably herein, and refer to any animal, or cells thereof whether in vitro or in situ, amenable to the methods described herein. In certain non-limiting embodiments, the patient, subject or individual is a human. The term “chimeric antigen receptor (CAR)”, as used herein, refers to a fused protein comprising an extracellular domain capable of binding to a predetermined antigen, an intracellular segment comprising one or more cytoplasmic domains derived from signal transducing proteins different from the polypeptide from which the extracellular domain is derived, and a transmembrane domain. The term “efficient amount”, as used herein, refers to the amount of an active agent (such as one or more compounds provided herein alone, in combination, or potentially in combination with other therapeutic agent(s)) sufficient to induce a desired biological result. That result may be amelioration or alleviation of the signs, symptoms, or causes of a cancer-related disease, or any other desired alteration of a biological system.

### EXAMPLES

### Cell Culture Media

For the culture of primary human CD8 T cells, PHA-induced T cell blasts, Jurkat T cells, PC3-PIP and T2 cells, RPMI-1640 medium (Invitrogen) was supplemented with heat-inactivated 10% fetal calf serum (HI FCS, Gibco), 50 U ml⁻¹ penicillin (Invitrogen) and 50 μg ml⁻¹ streptomycin (Invitrogen). Human REP T cells were expanded in AIM V medium (Thermo Fisher) mixed 1:1 with RPMI-1640 (Invitrogen) supplemented with 10% human HI AB serum, 50 U ml⁻¹ penicillin (Invitrogen) and 50 μg ml⁻¹ streptomycin, 1 mM pyruvate (Gibco), 1% MEM Non-Essential Amino Acids (Gibco), 1% GlutaMAX (Gibco) and 3,000 U ml⁻¹ human recombinant IL-2 (Proleukin, Novartis). Murine T cells and EL4 cells were kept in RPMI-1640 medium containing 10% HI FCS, 100 U ml⁻¹ penicillin, 100 μg streptomycin, 0.29 mg ml⁻¹ L-glutamine, 50 μM 2-Mercaptoethanol (Invitrogen). 293T human embryonic kidney (HEK-293T) were cultured in RPMI-1640 supplemented with 10% HI FCS, 2 mmol lglutamine, 100 μg ml⁻¹ penicillin and 100 U ml⁻¹ streptomycin (all purchased from Invitrogen). MC38-OVA cells were maintained in RPMI-1640-Glutamax medium supplemented with 10% FCS, 50 U mL⁻¹ penicillin and 50 μg mL⁻¹ streptomycin, 1 mM sodium pyruvate, 50 μM 2-Mercaptoethanol and under geneticin selection (0.4 mg mL⁻¹ G418). All reagents were purchased from Gibco. Magnesium-free medium was self-made with double distilled water (ddH₂O), supplemented according to manufacturer's instruction with RPMI-1640 amino acid solution (Sigma Aldrich), RPMI-1640 vitamin solution (Sigma Aldrich), 1% GlutaMAX (Gibco), 25 mM HEPES (Gibco), 2 g L⁻¹ sodium bicarbonate (Sigma Aldrich), 2 g L⁻¹ glucose (Sigma Aldrich), 100 mg L⁻¹ calcium nitrate (Sigma Aldrich), 400 mg L⁻¹ potassium chloride (Sigma Aldrich), 6 g L⁻¹ sodium chloride (Sigma Aldrich), 800 mg L⁻¹ sodium phosphate dibasic (Sigma Aldrich), 1 mg L⁻¹ Glutathion (Sigma Aldrich), 50 U ml⁻¹¹ penicillin and 50 μg ml⁻¹ streptomycin and 10% HI dialyzed FCS (dFCS, Gibco). For functional readouts, the medium was either supplemented, as indicated, with 1.2 mM MgCl₂ or 1.2 mM MgSO₄, or left untreated (=0 mM Mg²⁺). Cells of every condition were washed initially twice in magnesium-free medium prior to any functional read out. Low background Mg²⁺ values in self-made medium was verified by ICP-MS (data not shown).

### Cell Lines

Jurkat T cells (Clone E61, TIB-152) and HEK-293T were purchased from ATCC. T2 cells and EL4 were kindly provided by Prof. Zippelius (University of Basel). MC38-OVA were originally provided by Pedro Romero (University of Lausanne). PC3-PIP cell lines were originally provided by A. Rosato (University of Padua, Padova). Cells were cultured as described above.

### Mice

C57BL/6, MHC class I-restricted OVA-specific T cell receptor (OT-I) transgenic and B6.129S7-Itgaltm1B11J (LFA-1 KO) mice were originally purchased from Jackson Laboratories (USA) and thereafter bred and housed at specific pathogen free (SPF) conditions at the University of Basel. Age and sex matched C57BL/6 mice were purchased from Charles River (Italy) for intratumoral Mg²⁺-application experiments. Mice were maintained at SPF conditions and acclimatized for 1 week prior to experiments at the animal facility of the University of Geneva. All experiments were conducted in accordance to the Swiss Federal Veterinary Office guidelines and were approved by the Cantonal Veterinary Office (Canton of Basel-Stadt and Geneva). All cages provided free access to food and water. During experimentation, all animals were monitored at least every other day for signs of distress and, if required, body weight was measured three times a week. Mice were killed at the endpoint by carbon dioxide overdose.

### Human Naïve and Memory T Cell Isolation

Blood samples were obtained from healthy male and female donors (18-65 years old) as buffy coats after written informed consent (Blood donor center, University Hospital Basel).

Peripheral blood mononuclear cells (PBMCs) were isolated by standard density-gradient centrifugation protocols (Lymphoprep; Fresenis Kabi). MACS beads and LS columns (both Milteny Biotec) were used to sort CD8 positive T cells. The positively selected CD8 T cells were incubated with APC anti-CD62L mAb (ImmunoTools) and Pacific Blue anti-CD45RA (Beckman Coulter). Naive and EM CD8⁺ T cells were identified as CD62L⁺ CD45RA⁺ and CD62L⁻ CD45RA⁻ populations, respectively. Cell sorting was performed with a BD FACSAria III or BD influx cell sorter (BD Bioscience). Cells were rested for 24 h at 37° C. prior to further experiments.

### Generation of Human T Cell Blasts (PHA-Blasts)

PBMCs were activated with 10 μg ml⁻¹ Phytohaemagglutinin (PHA, Thermo Fisher) and 300 U ml⁻¹ human recombinant IL-2 (Proleukin, Novartis). PHA-blasts were expanded by adding fresh IL-2 every 3-4 days.

### In Vitro Activation Human T Cells

Unless stated otherwise, human EM CD8 T cells and PHA-blasts were activated in presence of plate-bound anti-CD3 Ab (HIT3a, Biolegend) at 1 μg ml⁻¹ and soluble anti-CD28 Ab 5μg ml⁻¹. Naïve CD8 T cells were activated with in house generated anti-CD3/anti-CD28 coated microbeads. Polybead microspheres (4.5 mm, Polyscience Eppenheim) were incubated with 1 μg anti-CD3 Ab and 10 μg anti-CD28 Ab. T cells were plated at 2×10⁵ cells per well in flat bottom 96 well plates (Greiner BIO One) in self-made medium supplemented with 10% dFCS and indicated supplementation of Mg²⁺ or LFA-1 inhibitor. Primary human T cells were activated for 24 h and PHA-blasts for 4 h.

### NY-ESO Peptides

NY-ESO-9c peptide (SLLMWITQC) was purchased in >95% purity from EZ Biolabs. Lyophilized peptides were resuspended at 10 mM in sterile dimethyl sulfoxide (DMSO) and stored at −20° C. until further use.

### T Cell Receptor Construct for REP T Cells

The lentiviral construct encoding for the codon-optimized WT LAU155 NY-ESO-1 T cell receptor alpha and beta chains under an hPGK promotor separated by an IRES domain was kindly provided by Dr. Michael Hebeisen and Dr. Natalie Rufer at the University of Lausanne (Hebeisen et al., 2013; Schmid et al., 2010). This TCR has a K_{D}=21.4 μM for its endogenous NY-ESO-1 SLLMWITQC peptide.

### Generation of Lentivirus for REP T Cells

To generate lentivirus, 2.5×10⁶ low passage HEK293T cells were cultured in DMEM medium (Thermo Fisher) and seeded into a 15 cm tissue-culture treated dish. After 3 days, 2^{nd} generation LTR-containing donor plasmid, packaging plasmid pCMV-delta8.9 and the envelope plasmid VSV-G were mixed at a 4:2:1 ratio in unsupplemented Opti-MEM (Thermo Fisher) and sterile filtered. This solution was then mixed with polyethyleneimine 25 kDa (Polysciences Inc.), also diluted in Opti-MEM at a DNA:PEI ratio of 1:3. 28 μg of DNA was transfected per 15 cm dish.

After 2 days, supernatants were collected from cells (exchange medium) and filtered through a 0.45 μm PES filter. Supernatants were stored for 1 day at 4° C. until the second batch of supernatant was collected 24 h later. The supernatant containing lentiviral particles was concentrated by ultra-centrifugation at 40,000×g for 2 h at 4° C., resuspended in 0.1% BSA in PBS, and frozen to −80° C.

### Transduction of Human T Cells for REP T Cell Production

To generate NY-ESO-1 TCR specific T cells, human healthy donor PBMCs were thawed and washed in PBS. CD8 T cells were then isolated using the CD8 microbeads (Miltenyi) according to the manufacturer's instructions on an AutoMACS (Myltenyi). Isolated cells were washed and resuspended in medium supplemented with 150 U ml⁻¹ IL-2 and plated at 1.5 mio ml⁻¹. CD8 T cells were then activated at a 1:1 ratio with activation beads from T cell activation and expansion kit (Miltenyi) according to manufacturer's instructions. 24 h later, NY-ESO-1 TCR lentiviral particles, produced as described above, were added at a multiplicity of infection (MOI) of 2. Cells were then expanded every 2 days with fresh medium and replenishing 50 Uml⁻¹ IL-2 for 5 days. NY-ESO-1 TCR positive T cells were sorted with FACSAria III or FACS SorpAria (BD) and re-stimulated with NY-ESO-9c peptide. A cell density of 0.5-2×10⁶ cells ml⁻¹ was maintained for expansion and 3,000 U ml⁻¹ IL-2 replaced ever third day. After 1 week of expansion, cells were either stored in liquid nitrogen or further expanded and subsequently used for functional read out as described below.

### Activation and Cytotoxicity of REP T Cells

REP T cells were incubated with T2 target cells in flat bottom 96 well-plate, if not indicated otherwise, at a 1:1 ratio (4-6×10⁴ each). Optimal ratio had been titrated for each donor beforehand. In order to distinguish the different cell populations, REP T cells were labeled with CellTrace Violet (CTV, Invitrogen) and T2 target cells with carboxyfluorescein diacetate succinimydyl ester (CFSE, Invitrogen). Prior to co-incubation, CFSE-labeled T2 target cells were pulsed with NY ESO peptides at 10⁻⁸ M for 30 min in magnesium-free medium and were washed three times before being re-suspended with REP T cells in magnesium-free medium supplemented with 10% dFCS at indicated cation or LFA-1 inhibitor concentration. For all co-incubation experiments, cells were allowed to sediment without centrifugation. For degranulation assays, an anti-CD107a-AF647 Ab was added directly into culture medium throughout the entire co-incubation. After 4 hours, cells were harvested, washed in cold FACS Buffer and gently fixed with PFA 2% for 15 min at room temperature. Cytotoxicity was examined with NucView 488 fluorogenic caspase-3 substrate (Biotium). Fluorogenic caspase substrate was added to wells at the beginning of co-incubation at final concentration of 1 μM.

After 45 min, cells were washed in FACS Buffer and gently fixed with PFA 2% for 15 min at room temperature. For analysis of protein phosphorylation, co-incubation was terminated after 25 min as described in Flow cytometry section below. Alternatively, luciferase-expressing T2 target cells were used (FIG. 4e). For this particular experiment, REP T cells T2 target ration was 2:1. CBR-LFA1/2 (Biolegend) or isotype control (Biolegend) were added upon start of co-culture at a final concentration of 10 μg ml⁻¹. Cytotoxicity was quantified after adding luciferin at 0.15 mg ml⁻¹ (PerkinElmer) to medium and measuring luminescent signal intensity by plate reader (Synergy H1, BioTek).

### Recombinant Lentivirus Production for CAR T Cells

High-titer replication-defective lentivirus was produced and concentrated by ultracentrifugation for primary T-cell transduction. Briefly, 24 h before transfection, HEK293 cells were seeded at 10×106 in 30 ml of medium in a T-150 tissue culture flask. All plasmid DNA was purified using the Endo-free Maxiprep kit (Invitrogen, Life Technologies). HEK-293T cells were transfected with 7 μg pVSV-G (VSV glycoprotein expression plasmid), 18 μg of R874 (Rev and Gag/Pol expression plasmid) and 15 μg of pELNS transgene plasmid, using a mix of Turbofect (Thermo Fisher) and Optimem medium (Invitrogen, Life Technologies, 180 μl of Turbofect for 3 ml of Optimem). The viral supernatant was collected 48 h after transfection. Viral particles were concentrated by ultracentrifugation for 2 h at 24,000 g and resuspended in 400 μl medium, followed by immediate snap freezing on dry ice.

### Primary Human T-Cell Transduction for CAR T Cell Generation

Primary human T cells were isolated from the peripheral blood mononuclear cells of healthy donors (HDs; prepared as buffycoats or apheresis filters). All blood samples were collected with informed consent of the HDs, and genetically engineered with ethics approval from the Canton of Vaud. Total peripheral blood mononuclear cells were obtained via Lymphoprep (Axonlab) separation solution, using a standard protocol of centrifugation. CD4 and CD8 T cells were isolated using a magnetic bead-based negative selection kit following the manufacturer's recommendations (easySEP, Stem Cell technology). Purified CD4 and CD8 T cells were cultured at a 1:1 ratio and stimulated with anti-CD3 and anti-CD28 Ab-coated beads (Invitrogen, Life Technologies) at a ratio of 1:2 T cells to beads. T cells were transduced with lentivirus particles at 18-22 h after activation. Human recombinant IL-2 (hIL-2; Glaxo) was replenished every other day for a concentration of 50 IU ml⁻¹ until 5 days after stimulation (day +5). At day +5, magnetic beads were removed, and h-IL-7 and h-IL-15 (Miltenyi Biotec) were added to the cultures at 10 ng ml⁻¹ replacing h-IL-2. A cell density of 0.5-1×10⁶ cells ml⁻¹ was maintained for expansion. Rested engineered T cells were adjusted for equivalent transgene expression before all functional assays.

Cytotoxicity Assay with CAR T Cells

Cytotoxicity assays were performed using the IncuCyte Instrument (Essen Bioscience). Briefly, 1.25×10⁴ PC3-PIP target cells were seeded in flat bottom 96-well plates (Costar, Vitaris). Four hours later, rested T cells (no cytokine addition for 48 h) were washed and seeded at 2.5×10⁴ per well, at a 2:1 effector to target ratio in self-made medium supplemented with 10% dFCS and 0.6 mM MgCl₂ or without Mg²⁺ supplementation. No exogenous cytokines were added during the co-culture period of the assay. IncuCyte Caspase-3/7 (Essen Bioscience) was added at a final concentration of 5 μM in a total volume of 200 μl. Internal experimental negative controls were included in all assays, including co-incubation of untransduced (UTD)-T cells and tumor cells in the presence of IncuCyte Caspase-3/7 reagent to monitor spontaneous cell death over time. As a positive control, tumor cells alone were treated with 1% triton solution to evaluate maximal killing in the assay. Images of total green area per well were collected every 2 h of the co-culture. The total green area per well was obtained by using the same analysis protocol on the IncuCyte ZOOM software provided by Essen Bioscience. All data were normalized by subtracting the background fluorescence observed at time 0 (before any cell killing by CAR-T cells) from all further time points.

### Cytokine Release Assay of CAR T Cells

Cytokine release assays were performed by co-culture of 5×10⁴ T cells with 5×10⁴ target cells per well in 96-well round-bottom plates, in duplicate, in a final volume of 200 μl of self-made medium supplemented with 10% dFCS and 0.6 mM MgCl₂ or without Mg²⁺ supplementation. After 24 h, the co-culture supernatants were collected and tested for the presence of IFNγ by commercial enzyme-linked immunosorbent assay kits according to the manufacturer's protocol (BioLegend).

### Murine MC38-OVA Tumor Model—Set Up

Unless stated otherwise, 6 to 12 weeks old female mice were used for experiments. In assays with pre-immunized mice, mice were immunized 19 days before tumor implantation by subcutaneous injection of 100 μg of OVA protein (Invivogen) and 50 μg of CpG-B ODN 1826 (Eurogentec), resuspended in 100 μL of PBS. For tumor implantation, mice were inoculated subcutaneously onto the flanks with 0.5×10⁶ MC38-OVA cells, resuspended in 100 μL of PBS. In bilateral tumor experiments, mice received 50 μL intra-tumoral injections of either 3 mM NaCl or 3 mM MgCl₂ (both diluted in ddH2O). Injections of NaCl solution was applied in left flank tumor, whereas MgCl2 solution was injected in contralateral tumor. I.t. injections were initiated once tumors were palpable, usually between day 5 and 10 after tumor injection. Injections were repeated every third day. Tumor size was quantified using a caliper and tumor volume was calculated using a rational ellipse formula (α2×β×π6, α being the shorter axis and β the longer axis). In all survival experiments, mice were withdrawn from the study after any tumor dimension had reached a length greater than 15 mm.

### Murine MC38-OVA Tumor Model—In Vivo CD8⁺ T Cell Depletion

For CD8 depletion experiment, mice were immunized with OVA, as described above, and inoculated with 0.5×10⁶ MC38-OVA cells unilaterally on the flank. Intratumoral injections of either 3 mM NaCl or 3 mM MgCl2 were initiated, and repeated every third day as tumors became palpable. CD8 T cells were depleted by administering anti-CD8a Ab (53-6.72, BioXCell) at 10 mg kg⁻¹ i.p. once per week.

### Murine MC38-OVA Tumor Model—In Vivo PD-1 Blockade

For PD-1 blockade experiments, mice were immunized with OVA, as described above, and inoculated with 0.5×10⁶ MC38-OVA cells unilaterally on the flank. As tumors became palpable—at day 5—intratumoral injections of either 3 mM NaCl or 3 mM MgCl2 were initiated, and repeated every third day for 8 cycles. Mice were additionally injected i.p. with isotype control (IgG2a) or anti-PD-1 Ab on day 9, 12, and 15 post-tumor implantations, at a dose of 200 μg per mouse diluted in 100 μL of pH-matched PBS (according to manufacturer's recommendations). The antibodies used were: anti-PD-1 IgG2a Ab (clone RMP1-14) or IgG2a isotype control Ab (clone 2A3, both purchased from BioXCell).

### Murine MC38-OVA Tumor Model—Flow Cytometry Analysis of Tumor-Infiltrating Immune Cells

Tumor tissue was isolated from mice, weighed and minced using razor blades. Tissue was then digested using accutase (PAA), collagenase IV (Worthington), hyaluronidase (Sigma), and DNAse type IV (Sigma) for 60 min at 37° C. with constant shaking. The cell suspensions were filtered using a cell strainer (70 μm). Precision Counting beads (Biolegend) were added before staining to quantify the number of cells per gram of tumor. Single cell suspensions were blocked with rat anti-mouse FcγIII/II receptor (CD16/CD32) blocking antibodies (‘Fc-block’) and stained with live/dead cell-exclusion dye. Cells were then incubated with fluorophore-conjugated antibodies directed against cell surface antigens, washed and resuspended in FACS buffer (PBS+2% FBS). For intracellular/intranuclear antigens, cells stained with cell surface antibodies were fixed and permeabilized using Foxp3/transcription factor staining buffer (eBioscience) prior to incubation with antibodies directed against intracellular antigens.

### Magnesium Restricted Diet

Magnesium restricted diet and matching control diet, based on the purified ingredient rodent diet AIN-76A, were purchased at Research Diets Inc. (USA).

### Murine CTL Differentiation and Cultivation

Single cell suspensions were made from lymph nodes and spleens harvested from C57B1/6 and LFA-1 KO mice (male and female, 6-10 weeks, equal distribution of sex and age). Naive CD8 T cells were isolated using a magnetic bead-based negative selection kit following the manufacturer's recommendations (easySEP, Stem Cell technology). Naïve T cells (2×10⁵ per well) were plated in presence of 5 μg anti-CD3 Ab (plate-bound) and 1 μg anti-CD28 Ab (soluble; both from Biolegend) for 2 days in presence 100 U ml⁻¹ of IL-2 (Proleukin). Cells were washed and seeded in fresh medium at 10⁶ ml⁻¹ in round bottom 96 well-plates with 500 U ml⁻¹ IL-2. A cell density of 0.5-2×10⁶ cells ml⁻¹ was maintained for expansion and IL-2 was replaced on a daily basis. Functional read outs were carried out 7-19 days after initial activation and in the absence of IL-2.

### CRISPR-Cas9 Editing of Murine OT-1 Cells and Human Jurkat T Cells

crRNAs were selected from predesigned CRISPR-Cas9 guide RNAs Tool from IDT. Product ID and sequences are listed in Supplemental Table I. crRNA (IDT) or negative control crRNA #1 (IDT) and trRNA (IDT) were mixed at a 1:1 ratio to a final concentration of 50 μM in nuclease-free duplex buffer (IDT), annealed at 95° C. for 5 min and added to 40 μM Cas9 (QB3 MacroLab, UC Berkeley) followed by incubation at room temperature for at least 10 min. Murine OT-1 cells were transfected with the Mouse T Cell Nucleofector Kit (Lonza) according to manufacturer's instructions using 2b Nucleofector. Briefly, single cell suspensions were made from lymph nodes and spleens harvested from OT-I mice (male and female, 6-10 weeks, equal distribution of sex and age). 2×10⁶ OT-I lymphocytes were resuspended in 100 μl of Nucleofector Solution and combined with 20 μM RNP. An appropriate nucleofector program was applied. Cells rested in Mouse T Cell Nucleofector Medium (Lonza) for 24 h and were then activated with OVA₂₅₇₋₂₆₄ peptide pulsed (10⁻⁹ M) C57/B16 splenocytes for 3 days in presence 100 U ml⁻¹ of IL-2 (Proleukin). Cells were washed and seeded in fresh medium at 10⁶ ml⁻¹ in round bottom 96 well-plates with 500 U ml¹ IL-2. Knock-out efficiency was validated by flow cytometry and purified by cell sorting.

Jurkat T cells were transfected as described above using the AMAXA cell line V nucleofection kit (Lonza). Knock-out efficiency was validated by flow cytometry and purified by cell sorting. Jurkat T cells were initially expanded for 1 week and then stored in liquid nitrogen.

### In Vitro Activation Murine CTLs

CTLs of WT or LFA-1 KO C57/B16 were activated, unless stated otherwise, in presence of plate-bound anti-CD3 Ab (145-2C11, Biolegend) at 0.05 μg ml⁻¹ and soluble anti-CD28 Ab (37.51, Biolegend) at 1 μg ml⁻¹ at 2×10⁵ cells per well in a flat bottom 96 well plate for 8 h, if not stated otherwise. Staining for surface activation markers is described below. Cytotoxicity was evaluated with NucView 488 fluorogenic caspase-3 substrate. CTV-labelled CTLs and CF SE-labeled EL4 target cells were incubated in presence of PHA at indicated concentrations for 4 hours in a flat bottom 96 well plate. Caspase-3 substrate was added for final 45 min of incubation. Cells were harvested, washed in FACS Buffer and gently fixed with PFA 2% for 15 min at room temperature.

OT-I derived CTLs were stimulated with OVA₂₅₇₋₂₆₄ peptide (SIINFEKL, Eurogentec) or the altered peptide ligands R7 (SIIQFERL, Eurogentec), H7 (SIIQFEHL, Eurogentec) or G4 (SIIGFEKL, Eurogentec) at 10 μM for 4 h. Cells were harvested and stained for surface activation markers. For cytotoxicity assays, EL4 target cells were pulsed with different OVA peptides at 1 μM for 30 min prior to co-incubation. Cytotoxicity was either evaluated with fluorogenic caspase-3 substrate (as described above) or luciferase-expressing EL4 target cells. Cytotoxicity was quantified after adding luciferin at 0.15 mg ml⁻¹ (PerkinElmer) to medium and measuring luminescent signal intensity by plate reader (Synergy H1, BioTek).

### Cytometric Bead Array (CBA)

Cytokine concentrations in cell culture supernatants were determined using the LegendPlex cytrometric bead Array human Th1-Pannel (Biolegend) according to manufacturer's instructions.

### Metabolic Assays

A Seahorse XF-96e extracellular flux analyzer (Seahorse Bioscience, Agilent) was used to determine the metabolic profile of cells. T cells were plated (2×10⁵ cells/well) onto Celltak (Corning, USA) coated cell plates. Experiments were carried out in unbuffered, serum- and Mg²⁺-free self-made medium. Medium was reconstituted with ±1.2 mM MgCl₂. Reconstitution of Mg²⁺ was either present from beginning of experiment or applied onto plated cells via the instrument's multi-injection port. All following concentration represent final well concentrations of indicated substance. Human T cells were activated by injection anti-CD3 Ab (1 μg mL⁻¹), or anti-CD3 Ab (1 μg mL⁻¹) and anti-CD28 Ab (10 μg mL⁻¹). In certain experiments (as indicated) anti-CD3/CD28 antibodies were cross-linked with additional injection of secondary goat anti-mouse Ab (5 μg mL⁻¹, Thermo Fisher). Murine T cells were activated by injection anti-CD3 Ab (5 μg mL⁻¹) and anti-CD28 Ab (2.5 μg mL⁻¹).

### Calcium Flux Assay

Jurkat T cells were loaded with Fluo4 (Invitrogen) at a final concentration of 2 μM in Mg²⁺ free self-made medium for 30 min at 37° C. Cells were washed twice and plated at 2×10⁵ per well in a black flat bottom 96 well-plate (Greiner BIO one) which had been precoated with collagen (Thermo Fisher) to enhance cell attachment. An additional incubation for 15 min at 37° C. allowed cells to adhere and Fluo4 probe to de-esterified completely. Jurkat T cells stimulated with 10 μg ml⁻¹ anti-CD3. Fluorescence intensity over time was measure with a Tecan Spark M10 plate reader. Samples were run in technical duplicates and the mean of fluorescent signal intensity was normalized to unstimulated baseline values.

Co-Culture Assay with Blinatumomab

Blinatumomab (Amgen) was derived from the leftover if infusions. Human PHA-blasts were incubated with Ramos target cells in flat bottom 96 well-plate at a 0.5:1 ratio (6.5×10⁴ PHA blasts and 1.3×10⁵ Ramos cells) at indicated Blinatumomab concentrations. In order to distinguish the different cell populations, PHA-blasts were 49abelled with CTV and T2 target cells with CFTR Invitrogen. For all co-incubation experiments, cells were allowed to sediment without centrifugation. For quantification of LFA-1 conformation, m24 was directly added to the cell culture medium and incubated for 10 min followed by incubation for 30 min on ice before washing and subsequent fixation with 2% PFA. Cytotoxicity was quantified after 3.5 h with CellEvent Caspase-3/7 Green Detection Reagent (Invitrogen, Thermo Fisher) as described above. Caspase substrate was added for final 45 min of incubation at final concentration of 2 μM. Cells were harvested, washed in FACS Buffer and fixed with PFA 2% for 15 min at RT prior to analysis by FACS.

### Recombinant Lentivirus Production for Anti-CD19 CAR T Cells

24 h before transfection, HEK-293T cells were seeded (3.8×106 cells 10 ml⁻¹ media). All plasmid DNA was purified using the Endotoxin-free Plasmid Maxiprep Kit (Sigma). HEK-293T cells were transfected with 1.3 pmol psPAX2 (lentiviral packaging plasmid) and 0.72 pmol pMD2G (VSV-G envelope expressing plasmid) and 1.64 pmol of pCAR-CD19CAR-p2a-EGFP (Creative Biogene) using Lipofectamine 2000 (Invitrogen) and Optimem medium (Invitrogen, Life Technologies). The viral supernatant was collected 48 h after transduction. Viral particles were concentrated using PEG precipitation and stored at −80° C.

### Primary Human T Cell Transduction for Anti-CD19 CAR T Cell Generation

Blood samples (Blood donor center, University Hospital Basel) were obtained from healthy donors after written informed consent. PBMCs were isolated by standard density-gradient centrifugation protocols (Lymphoprep; Fresenius Kabi). CD4⁺ and CD8⁺ T cells were positively selected using magnetic CD4⁺ and CD8⁺ beads (Miltenyi Biotec). Purified CD4⁺ and CD8⁺ T cells were cultured in R10AB. CD4⁺ and CD8⁺ T cells were plated into a 24-well cell culture plate and stimulated with anti-CD3 and anti-CD28 monoclonal antibody-coated beads (Miltenyj, T cell activation & expansion kit) in a ratio of 1:1 in medium containing IL-2 (150 U ml−1). T cells were transduced with lentiviral particles at 18-22 h after activation in media containing Polybrene (6 μg ml−1, Millipore). Every second day medium was replaced with fresh IL-2 (150 U ml−1). Five days after transduction GFP⁺ cells were sorted enrich CD19-CAR⁺ cells and magnetic beads were removed from non-transduced cells. Cells were further expanded for 3 days in medium containing IL-2 (150 U ml−1) before the target cell killing assay.

Cytotoxicity Assay with Anti-CD19 CAR T Cells

CD8⁺ anti-CD19 CAR T-cells were incubated with Ramos target cells at a 0.1-0.33:1 ratio (0.5-1.5×10⁴ CART cells and 5×10⁴ Ramos Target cells). Ramos cells had been labelled with CFTR prior to co-incubation. Cells were allowed to sediment without centrifugation in flat bottom 96 well-plate and incubated for 3 h. Cytotoxicity was quantified by flow cytometry using BioTracker NucView 405 Blue Caspase-3 Dye (Sigma-Aldrich).

### Recombinant Lentivirus Production for Anti-PSMA CAR T Cells

High-titer replication-defective lentivirus was produced and concentrated by ultracentrifugation for primary T cell transduction. Briefly, 24 h before transfection, HEK-293 cells were seeded at 10×10⁶ in 30 mL of medium in a T-150 tissue culture flask. All plasmid DNA was purified using the Endo-free Maxiprep kit (Invitrogen, Life Technologies). HEK-293T cells were transfected with 7 μg pVSV-G (VSV glycoprotein expression plasmid), 18 μg of R874 (Rev and Gag/Pol expression plasmid) and 15 μg of pELNS transgene plasmid, using a mix of Turbofect (Thermo Fisher) and Optimem medium (Invitrogen, Life Technologies, 180 μl of Turbofect for 3 mL of Optimem). The viral supernatant was collected 48 h after transfection. Viral particles were concentrated by ultracentrifugation for 2 h at 24,000×g and resuspended in 400 μl medium, followed by immediate snap freezing on dry ice.

### Primary Human T Cell Transduction for Anti-PSMA CAR T Cell Generation

Primary human T cells were isolated from the peripheral blood mononuclear cells of healthy donors (HDs; prepared as buffycoats or apheresis filters). All blood samples were collected with informed consent of the healthy donors, and genetically engineered with ethics approval from the Canton of Vaud, Switzerland. PBMC were obtained via Lymphoprep (Axonlab) separation solution, using a standard protocol of centrifugation. CD4 and CD8⁺ T cells were isolated using a magnetic bead-based negative selection kit following the manufacturer's recommendations (easySEP, Stem Cell technology). Purified CD4 and CD8⁺ T cells were cultured at a 1:1 ratio and stimulated with anti-CD3 and anti-CD28 Ab coated beads (Invitrogen, Life Technologies) at a ratio of 1:2 T cells to beads. T cells were transduced with lentivirus particles at 18-22 h after activation. Human recombinant IL-2 (h-IL-2; Glaxo) was replenished every other day for a concentration of 50 IU mL−1 until 5 days after stimulation (day +5). At day +5, magnetic beads were removed, and h-IL-7 and h-IL-15 (Miltenyi Biotec) were added to the cultures at 10 ng mL⁻¹ replacing h-IL-2. A cell density of 0.5-1×10⁶ cells mL−1 was maintained for expansion. Rested engineered T cells were adjusted for equivalent transgene expression before all functional assays.

Cytotoxicity Assay with Anti-PSMA CAR T Cells

Cytotoxicity assays were performed using the IncuCyte Instrument (Essen Bioscience). Briefly, 1.25×10⁴ PC3-PIP target cells were seeded in flat bottom 96-well plates (Costar, Vitaris). Four hours later, rested T cells (no cytokine addition for 48 h) were washed and seeded at 2.5×10⁴ per well, at a 2:1 effector to target ratio in self-made medium supplemented with 10% dFCS and ±0.6 mM MgCl₂. No exogenous cytokines were added during the co-culture period. IncuCyte Caspase-3/7 (Essen Bioscience) was added at a final concentration of 5 μM in a total volume of 200 μl. Internal experimental negative controls were included in all assays, including co-incubation of untransduced (UTD)-T cells and tumor cells in the presence of IncuCyte Caspase-3/7 reagent to monitor spontaneous cell death over time. As a positive control, tumor cells alone were treated with 1% triton solution to evaluate maximal killing in the assay. Images of total green area per well were collected every 2 h of the co-culture. The total green area per well was obtained by using the same analysis protocol on the IncuCyte ZOOM software provided by Essen Bioscience. Cytotoxicity is reported as total area under the curve of the fluorescence driven by incorporation of cytotoxic green reagent in dead target cells (green area per μm2). All data were normalized by subtracting the background fluorescence observed at time zero (before any cell killing by CAR T cells) from all further time points.

### Cytokine Release Assay of Anti-PSMA CAR T Cells

Cytokine release assays were performed by co-culture of 5×10⁴ T cells with 5×10⁴ target cells per well in 96-well round-bottom plates, in duplicate, in a final volume of 200 μl of self-made medium supplemented with 10% dFCS and 0.6 mM MgCl₂ or without Mg²⁺ supplementation. After 24 h, the co-culture supernatants were collected and tested for the presence of IFN-γ by commercial enzyme-linked immunosorbent assay kits according to the manufacturer's protocol (BioLegend).

### Anti-PSMA CAR T Cell In Vivo Experiment

Male NSG mice of 10-12 weeks were put on Mg²⁺-restricted or matching control diet 5 days prior to tumor injection and kept on respective diet throughout the experiment. 5×10⁶ PC3-PIP tumor cells were injected subcutaneously. After 5 days, intravenous injection of saline solution or 2×10⁶ T cells (UTD or CAR T cells) were adoptively transferred intravenously. Tumor volume was monitored twice per week. The animals were monitored daily and the tumors were calipered every other day. Tumor volumes were calculated using the formula V=½(length×width2), where length is the greatest longitudinal diameter and width is the greatest transverse diameter determined via caliper measurement.

### Flow Cytometry

For analysis of surface markers, T cells were harvested at indicated time points, washed once in cold PBS and, if required, stained with Fixable Viability Dyes for 15 min at 4° C. Surface markers were stained with appropriate antibodies for 20 min at 4° C.

For evaluation of activation induced LFA-1 head-piece opening, T cells were activated for min and anti-human CD11a/CD18 (clone m24) was directly added in medium and incubated on ice for 20 min. Cells were then washed twice in FACS Buffer and fixed in 2% PFA, incubated at room temperature for 20 min and washed with FACS Buffer before acquisition.

For additional quantification of LFA-1 conformations, m24, KIM127 or TS2/4 mAbs were also directly added to the cell culture medium for 10 min at 37° C. and for 30 min on ice before washing and subsequent fixation with 2% PFA. For stainings with HI111, the cells were activated for 45 min, fixed with 2% PFA and subsequently stained with HI111 and washed with FACS Buffer before acquisition.

For intracellular TNF staining, cells were activated for 4 h as indicated. During the final 2 h of activation, cells were treated either with brefeldin A solution (BioLegend) to block cytokine secretion. Cells were then washed and fixed for 20 min at RT (fixation/permeabilization solution, BD Biosciences) and washed with permeabilization buffer (BD Biosciences) prior to staining for 45 min and further washing before acquisition. For analysis of protein phosphorylation, T cells were stimulated as indicated and fixed by adding 8% Paraformaldehyde (PFA) (Thermo Fisher) directly into the culture medium to obtain a final concentration of 4% PFA. Cells were incubated for 15 min at RT, washed with FACS buffer, followed by permeabilization with ice cold methanol at 4° C. for 5 min. fter washing with FACS buffer, cells were stained at room temperature for 30 min, washed and acquired.

BD Fortessa LSR II (BD Bioscience) or Cytoflex S (Beckmann) flow cytometer were used for flow cytometry

The following antibodies were used for staining:

### MC38-OVA Tumor Model:

CD3 (BUV805, BD Biosciences), CD4 (BUV496, BD Biosciences), CD8 (eFluor450, eBioscience), CD11b (APC-Cy7, BioLegend), CD11c (FITC, BioLegend), CD19 (BB515, BD Biosciences), CD25 (PE-Cy5.5, eBioscience), CD45 (BUV385, BD Biosciences), CD80 (BV605, BioLegend), CD103 (BV650, BD Biosciences), CD206 (BV711, BioLegend), CXCR3 (BUV737, BD Biosciences), F4/80 (AF647, BioLegend), FoxP3 (APC, eBioscience), GzmB (PE-eFluor610, Inivtrogen), Ki67 (AF532, eBioscience), LFA-1 (SB436, ThermoFisher), Ly-6G (BUV563, BD Biosciences), Ly-6c (PerCP, BioLegend), MHCII (BV510, BioLegend), NKp46 (BUV563, BD Biosciences), PD-1 (BV785, BioLegend), PD-L1 (BV421, BioLegend), TCF-7 (AF700, R&D Systems), Tim-3 (BB700, BD Biosciences), Zombie UV Fixable Viability Kit (BioLegend) Murine peritonitis model: CD8 (FITC, Biolegend), CD11b (PE-Cy5, Biolegend), CD11c (PE-Cy5, Biolegend), CD69 (APC, Biolegend), CD107a (PE/Cy7, Biolegend), B220 (PE-Cy5, Biolegend), F4/80 (PE-Cy5, Biolegend), Tetramer H-2 Kb OVA (PE, Tetramers core facility, University of Lausanne), Viability Dye (Zombie Red, BioLegend,)

### Murine In Vitro Activation:

CD11a (FITC and BV421, Biolegend), LFA-1 (BV421, Biolegend), CD8 (FITC, Biolegend), CD69, (APC, Biolegend), CD107a (PE-Cy7, Biolegend), Viability Dye (Aqua Zombie, Biolegend)

### Human T Cell In Vitro Activation

CD11a (FITC or unlabelled, Biolegend), CD18 (PE or unlabeled, Biolegend), CD18 (unlabeled, InVivo BioTech Services GmbH), CD25 (APC, BD), CD45RA (Pacific Blue, Beckmann), CD62L (APC, Immuno Tools), CD69 (PerCP, Biolegend), CD71 (PE, Biolegend), CD107a (AF647 BD and PE-Cy7 Biolegend), CD98 (FITC, BD Bioscience), m24 epitope LFA-1 (PE Biolegend), TCR Vbeta13.1 (FITC and PE-Cy7, Biolegend), TNF (PE, Biolegend), phospho-FAK (Tyr397, unlabeled, Thermo Fisher), Viability Dye (Aqua Zombie, Biolegend; Zombie Green, BioLegend), secondary goat anti-mouse AF488 (ThermoFisher)

### Chemicals

LFA-1 inhibitor studies were performed using BIRT377 at 50 μM (Tocris). All chemicals were aliquoted in DMSO and stored at —20° C.

### Statistical Analysis

Statistical significance was analyzed using Prism 8.0 (GraphPad Software, USA). P values of less than 0.05 were considered statistically significant.

crRNA Sequences

| Design ID | Species | Position | Strand | Sequence | PAM |
|---|---|---|---|---|---|
| Hs.Cas9. | Human | 30475350 | — | TGCCCG | AGG |
| ITGAL.1. | | | | ACTGGC | |
| AA | | | | ACTGAT | |
| | | | | AG | |
| Mm.Cas9. | Mouse | 127302137 | — | CACATAG | AGG |
| ITGAL.1. | | | | TTGATGG | |
| AB | | | | CACGAA | |

### EXAMPLE 1

### Intratumoral Magnesium Injections Improve Memory CD8 T Cell Mediated Antitumor Immunity

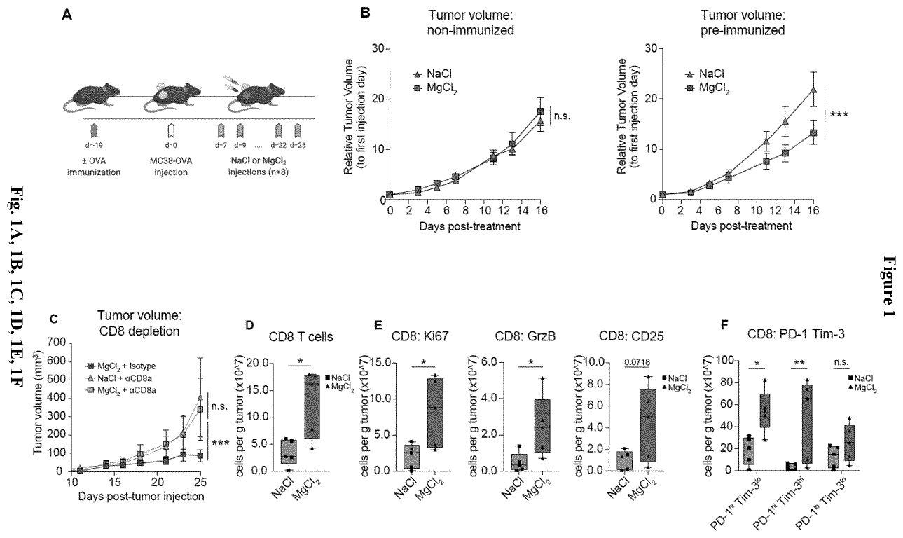

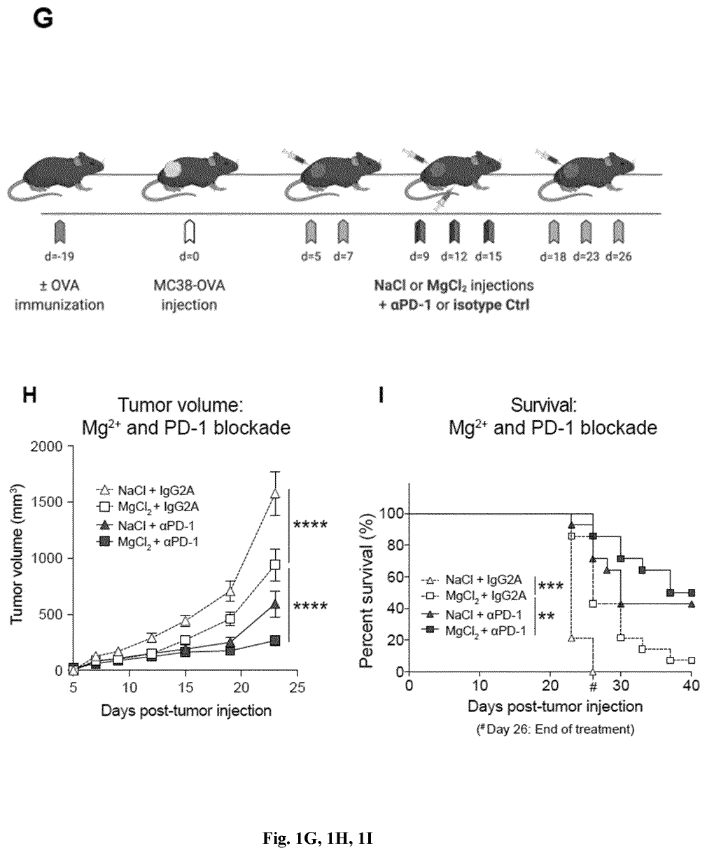

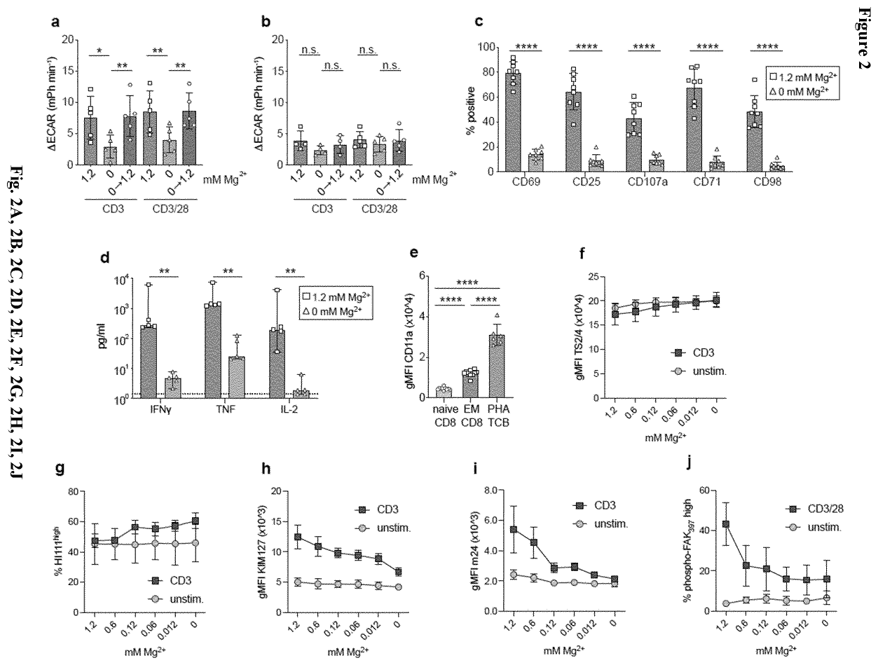

CD8 T cells are essential for antitumor immunity. To explore the role of Mg²⁺ in the tumor microenvironment, and specifically its functional impact on T cell immunity, we examined the effect of intratumoral (i.t.) Mg²⁺ administration in the MC38-OVA tumor model. Specifically, mice were either immunized against ovalbumin (OVA) or left untreated before subcutaneous implantation of OVA-expressing MC38 colorectal carcinoma cells bilaterally on the flanks. From day 7 onwards, right-side tumors were repeatedly injected with 3 mM MgCl₂ and left-side control tumors received 3 mM solution of NaCl (FIG. 1a, experimental scheme). While tumor growth was comparable between MgCl₂ and NaCl treatment in nonimmunized mice, intratumoral MgCl₂ administration significantly reduced tumor growth in pre-immunized mice indicating that increasing intratumoral Mg²⁺ concentrations augmented specifically memory T cell mediated antitumor immunity (FIG. 1b). OVA immunization induces OVA-specific memory T cells, including memory CD8⁺ T cells which play a key role in tumor rejection. and CD8 depletion experiments established that Mg²⁺ exerted its effect via CD8⁺ T cells (FIG. 1C). We thus sought to further define how i.t. Mg²⁺ affected the memory CD8⁺ T cell compartment. Using flow cytometry, we enumerated and phenotyped tumor-infiltrating immune cells. Notably, the number of tumor-infiltrating CD8⁺ T cells was increased in the Mg²⁺ treated group (FIG. 1D). Aligning with their increased number, more Mg²⁺ treated CD8⁺ T cells expressed Ki67 (FIG. 1E, left panel). In addition, more Mg²⁺ exposed CD8⁺ T cells contained Granzyme B and expressed the activation marker CD25 (FIG. 1E, middle and right panel). Further reflecting increased activation, PD-1 and TIM3 were also significantly more often (co-)expressed on Mg²⁺ treated CD8⁺ T cells (FIG. 1F).

Next, we examined whether the combination of MgCl₂ treatment with PD1 blockade could synergistically improve tumor suppression capacity of memory CD8⁺ T cells (FIG. 1G, Experimental scheme). Mice receiving intratumoral MgCl₂ in combination with PD-1 blockade were markedly superior at controlling tumor growth compared to other treatment regimens, with MgCl₂ alone improving immune control significantly (FIG. 1H). While intratumoral MgCl₂ application alone resulted in significantly improved animal survival compared to NaCl-treated control group, combining MgCl₂ with PD1 blockade resulted in additional survival benefit (FIG. 1I).

Taken together, our data demonstrated that intratumoral Mg²⁺ application potentiated antitumor activity of memory CD8 T cells and that increasing intratumoral Mg²⁺ concentration synergized with PD-1 blockade resulting in improved tumor suppression. All these experiments identified Mg²⁺ as an important modulator of memory CD8⁺ T cell-dependent tumor control

### EXAMPLE 2

### Extracellular Magnesium Enabled T Cell Activation of LFA-1^{high} T Cells Via LFA-1 Stabilization

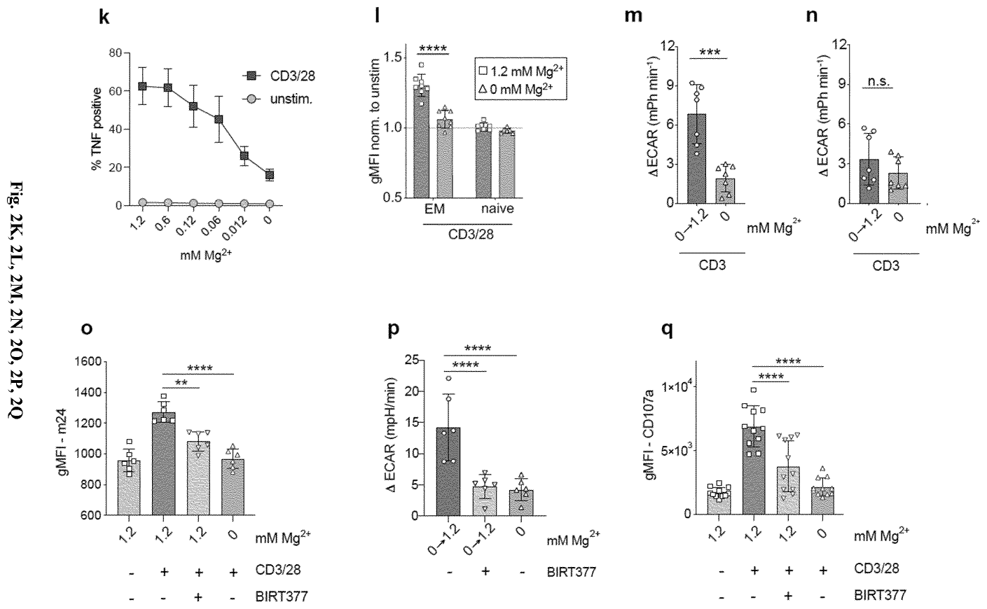

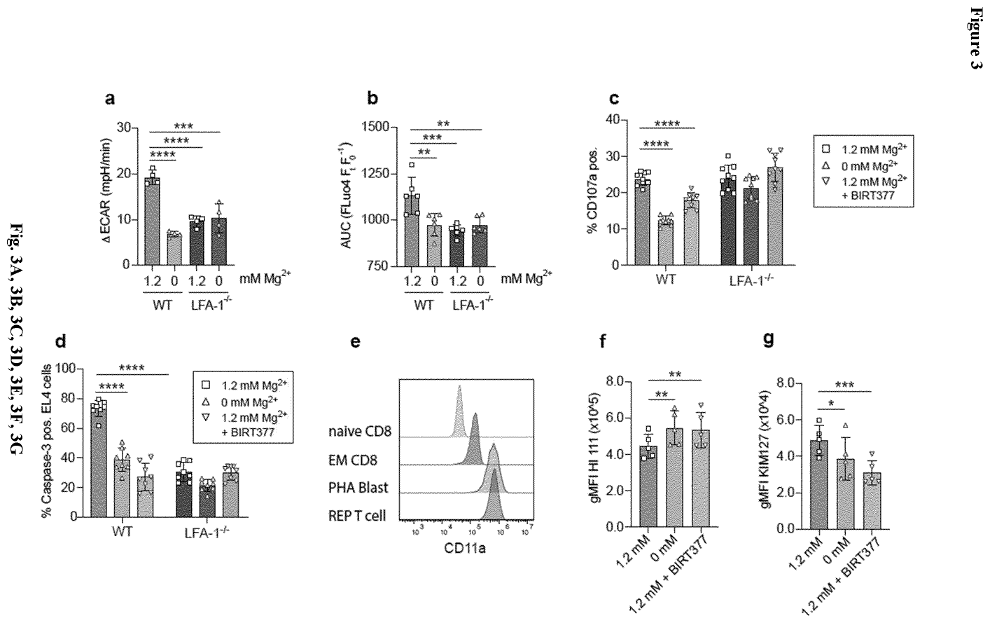

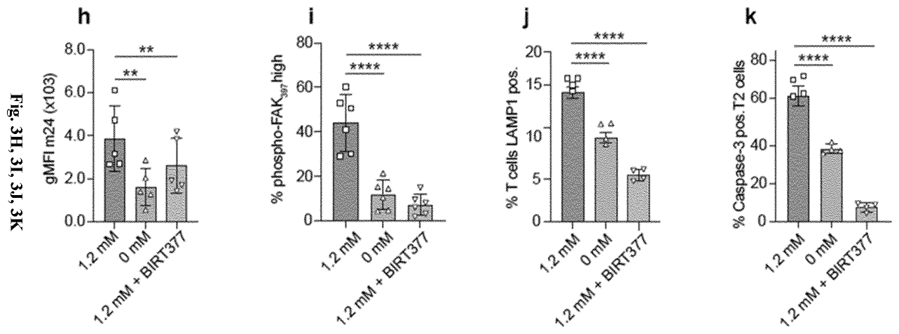

To determine whether the observed, memory cell-specific activation deficit in Mg²⁺-restricted conditions could be reproduced in vitro, we performed metabolic flux analysis with primary human effector-memory (EM) and naïve CD8 T cells. This method allows monitoring of T cell activation in real time as T cells exhibit an immediate upregulation of aerobic glycolysis upon activation—termed as ‘glycolytic switch’—enabling T cells to acquire effector capacity such as rapid production of IFNγ (Gubser P M, Bantug G R, Razik L, et al. Rapid effector function of memory CD8⁺ T cells requires an immediate-early glycolytic switch. Nat Immunol. 2013; 14(10):1064-1072. doi:10.1038/ni.2687). Analysis of glycolytic flux profiles revealed that glycolytic switching of EM CD8 T cells was blunted in absence of Mg²⁺. Notably, the activation deficit was independent of costimulation through CD28 and fully revertible upon add-back of Mg²⁺ just prior to activation (FIG. 2a). In contrast, naïve CD8 T cells showed no impaired activation-induced upregulation of glycolysis in absence of Mg²⁺ (FIG. 2b). These data indicated that (i) memory T cell specific impairment in Mg²⁺-restricted conditions—as found in previous in vivo experiments—could be reproduced in vitro, (ii) absence of extracellular Mg²⁺ affected proximal TCR signalling in EM CD8 T cells which in turn hindered glycolytic switching, and (iii) blunted glycolytic switching of EM CD8 T cells could be fully reversed by Mg²⁺ add-back shortly before activation arguing against irreversible cellular damage due to Mg²⁺ deprived conditions. Next, we assessed the expression of surface activation on EM CD8 T cells upon moderate TCR stimulation (plate bound anti-CD3 and soluble anti-CD28 Ab) for 24 h. In absence of extracellular Mg²⁺, EM CD8 T cells failed to upregulate T-cell activation markers such as indicators of early and late activation (CD69 and CD25, respectively); metabolic reprogramming (CD71, CD98), and degranulation (CD107a) (FIG. 2c). Measurement of cytokine secretion from these same assay wells revealed decreased production of IFNγ, TNF and IL-2 in Mg²⁺-restricted conditions (FIG. 2d). Analysis of CD11a surface expression on human naïve and EM CD8 T cells as well PHA-blasts revealed that naïve CD8 T cells had significant lower surface expression of CD11a compared to EM CD8 T cells or PHA-Blasts. PHA-blasts, on the other hand, exhibited the highest expression levels of CD11a (FIG. 2e). LFA-1 is known to have 3 conformational states: the bent conformation with closed headpiece, the extended conformation with closed headpiece and the extended conformation with open headpiece, which are corresponding to the low-, intermediate- and high-affinity states, respectively (Zhang K, Chen J. The regulation of integrin function by divalent cations. Cell Adh Migr. 2012; 6(1):20-29). On resting T cells, LFA-1 is predominantly in its inactive/bent confirmation and in response to TCR stimulation, LFA-1 converts from the low affinity to the high-affinity state. This transformation is coordinated by the metal-ion dependent adhesion site (MIDAS) which binds Mg²⁺ with high affinity. Therefore, the observation of memory CD8 T cells being Mg²⁺ dependent, while activation of naïve CD8 T cell seemed to be Mg²⁺ independent, is consistent given the differential LFA-1 expression pattern on T cell subpopulations rendering LFA-1^{high} more Mg²⁺ dependent. The mAb TS2/4 maps an epitope on CD11 a being present only in the assembled CD11a/CD18 heterodimer. The abundance of the TS2/4 epitope was independent on the extracellular Mg²⁺ concentration as well T-cell activation status (FIG. 2f). The mAb HI111 reports the inactive/bent LFA-1 conformation (FIG. 2g). PHA T-cell blast exhibited higher levels of inactive LFA-1 upon TCR-stimulation in decreasing, extracellular Mg²⁺-concentrations. Kim127 binds to an epitope on CD18 which is hidden in bent, inactive integrins and exposed upon integrin extension. We observed a dose-dependent KIM127 signal increase upon T-cell activation with increasing extracellular Mg²⁺ concentrations (FIG. 2h). The extended/open high affinity conformation of LFA-1 can be quantified by using the m24 antibody, e.g., by flow cytometry. In line with LFA-1 extension reported by KIM127, also head-piece opening was strongly depending on extracellular Mg²⁺ concentration (FIG. 2i). LFA-1 activation and subsequent outside-in signalling leads to phosphorylation of focal adhesion kinase (FAK). To probe this early LFA-1 downstream signal in relation to Mg²⁺ availability, we assessed the FAK phosphorylation in activated T cell blasts. In line with reduced LFA-1 extension as well head-piece opening in Mg²⁺-restricted conditions, FAK phosphorylation was decreased as well (FIG. 2j). Furthermore, assessment of activation-induced cytokine production exhibited a similar pattern of dose-dependence on extracellular Mg²⁺ concentration (FIG. 2k). EM CD8 T cells exhibited reduced activation induced LFA-1 head-piece opening in Mg²⁺-restricted conditions, while moderate TCR stimulation did not induce LFA-1 head-piece opening on naïve CD8 T cells in neither conditions (FIG. 2l). Moreover, using BIRT377 an allosteric LFA-1 inhibitor, stabilizing LFA-1 in its inactive, closed conformation, prevented activation-induced LFA-1 head-piece opening in presence of Mg²⁺ (FIG. 2o). Of note, LFA-1 activation did not differ between ±Mg²⁺ in unstimulated T cells (Data not shown). Inhibition of LFA-1 extension and head-piece opening in the course of T cell activation resulted in impaired glycolytic switching (FIG. 2p) as well decreased degranulation (FIG. 2q).

In all, these data indicated that reduced abundance of bent LFA-1 and subsequent extension as well head-piece opening, mediated by binding of Mg²⁺ to MIDAS, was crucial for the activation of LFA-1^{high} cells. The absence of Mg²⁺ in extracellular milieu, or pharmacologically forced stabilization of LFA-1 in bent/low-affinity confirmation resulted in blunted T cell activation. We therefore conclude that the activation of LFA-1^{high} T cells requires a mediator with moderate LFA-1 stabilization properties.

### EXAMPLE 3

### Magnesium Regulates Cytotoxic T Cell Activity via Moderate Modulation of LFA-1 Stabilization—Strong LFA-1 Stabilization Overrides Magnesium-LFA-1 Axis

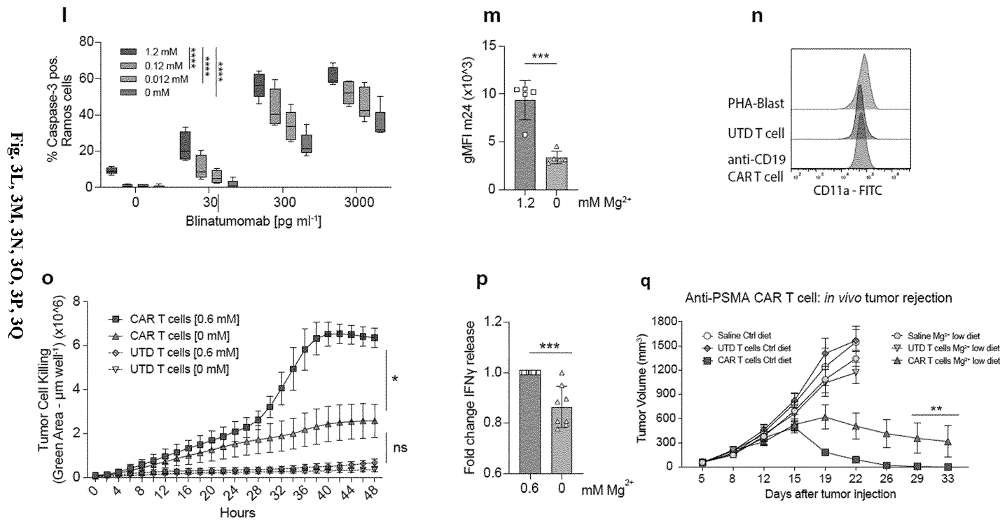

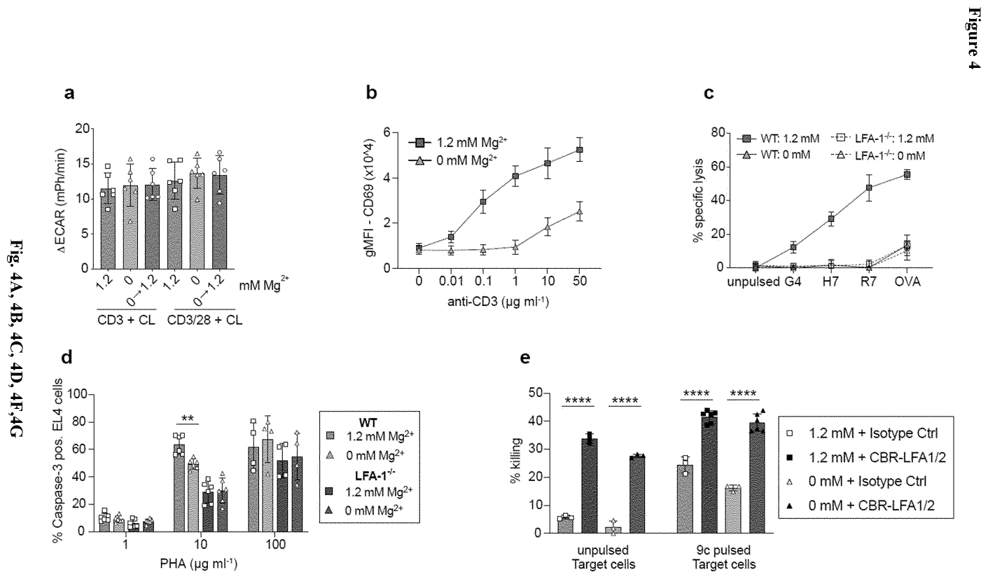

To elucidate whether extracellular Mg²⁺ mediates its T cell modulatory activity via LFA-1, we undertook our hypothesis genetic validation by conducting experiments with LFA-1-deficient (LFA-1^{-/-}) T cells. According to our previous findings, activation of LFA-1^{-/-} T cells was expected to be unaltered by extracellular Mg²⁺ concentrations. In a first attempt, we monitored activation induced glycolytic switching by metabolic flux analysis. While wildtype (WT) cytotoxic lymphocytes (CTLs) exhibited reduced glycolytic switching in Mg²⁺-depleted conditions, activation of LFA-1^{-/-} CTLs resulted in a reduced upregulation of aerobic glycolysis which was, indeed, independent from extracellular Mg²⁺ concentrations (FIG. 3a). Besides upregulation of aerobic glycolysis, TCR stimulation results also in rapid influx of extracellular calcium (Ca²⁺). Cytosolic Ca²⁺ is a pivotal second messenger and required for full T cell activation. In line with the metabolic flux analysis, depletion of extracellular Mg²⁺ concentrations resulted in decreased Ca²⁺ influx in WT Jurkat T cells while Ca²⁺ influx of LFA-1^{-/-} Jurkat T was reduced. Again, this reduction was independent from extracellular Mg²⁺ concentrations (FIG. 3b). Lack of LFA-1 functionality, resulting from either extracellular Mg²⁺ depletion or genetic deletion, reduced immediate Ca²⁺ influx but even more evidently resulted in absence of prolonged, sustained Ca²⁺ influx. Sustained Ca²⁺ influx is required for full T cell activation and has been reported to be mediated by translocation of mitochondria towards the immune synapse where they locally buffer high Ca²⁺ concentrations and thereby prolong opening of membrane Ca²⁺ channels (Quintana, A., Schwindling, C., Wenning, A. S., Becherer, U., Rettig, J., Schwarz, E. C., & Hoth, M. (2007). T cell activation requires mitochondrial translocation to the immunological synapse. Proceedings of the National Academy of Sciences, 104(36), 14418-14423). We therefore conclude that extracellular Mg²⁺—acting as an LFA-1 mediator with moderate LFA-1 stabilization properties—orchestrates the assembly of multimolecular signalling complexes at the immune synapse and thereby shapes T cell activation. We next assessed the importance of moderate LFA-1 stabilization for cytotoxic T cell activity. We therefore activated WT and LFA-1^{-/-} CTLs and evaluated degranulation by flow cytometry. Inhibition of LFA-1 functionality in WT CTLs by either Mg²⁺-restriction or pharmacological inhibition with BIRT377 resulted in reduced CTL degranulation. On the other hand, cytotoxic granule release in LFA-1^{-/-} CTLs was unaffected by such LFA-1 modulation (FIG. 3c). Also, cytotoxicity of WT CTLs, as assessed by frequency of apoptotic target cells, was reduced when LFA-1 functionality was blocked (FIG. 3d). LFA-1^{-/-} CTLs exhibited not only markedly decreased cytotoxic potential compared to WT CTLs, they were also unresponsive to LFA-1 modulation.

In order to generate sufficient numbers of tumor-specific T cells for patient administration, rapid expansion protocol (REP) can be used. REP T cells exhibit high LFA-1 surface expression (FIG. 3e). We co-cultured REP T cells with cognate-peptide pulsed tumor target cells and evaluated LFA-1 conformations. We observed reduced abundance of inactive, bent conformation in presence of extracellular Mg²⁺ (FIG. 3f) arguing for more active LFA-1 upon activation. In line with this finding, there was a significant increase of extended LFA-1 (FIG. 3g) with open head-piece (FIG. 3h). Also, FAK phosphorylation was significantly increased in presence of extracellular Mg²⁺ and subsequent degranulation (FIG. 3f) and cytotoxicity (FIG. 3g). LFA-1 inhibition, either by Mg²⁺-restriction or BIRT377 application, resulted in reduced degranulation or target cell killing (FIGS. 3f-k,). The use of blinatumomab (Blincyto®), a CD3/CD19 bispecific antibody engaging T cells to bind and eliminate CD19-positive cells, has improved the clinical outcome of B cell malignancies. Given its mode of action we aimed to test the impact of Mg²⁺ on blinatumomab efficacy. Blinatumomab-mediated cytotoxicity, in its reported therapeutic range (230-620 pg ml⁻¹), was strongly dependent on availability of Mg²⁺ (FIG. 3l) while LFA-1 head piece-opening was Mg²⁺-dependent as well.

The authors found that cytotoxic activity of chimeric antigen receptor (CAR) T cells, another adoptive cell-based immunotherapy exhibited also high surface expression of LFA-1 (FIG. 3n) and was also depending on extracellular Mg²⁺. Time-lapse killing assays revealed that Mg²⁺ restriction impaired the cytolytic activity of CAR T cells in vitro against tumor target cells (FIG. 3o and concomitantly reduced inflammatory IFNγ release (FIG. 3p). To probe whether reducing systemic Mg²⁺ via dietary restriction might affect CAR T cell mediated cytotoxicity in vivo, tumor rejection experiments were performed. Indeed, dietary Mg²⁺ restriction was sufficient to negatively impacted CAR T cell-mediated tumor-rejection, in vivo (FIG. 3q). These experiments highlighted that the efficacy of novel adoptive cell-based immunotherapies rely on moderate LFA-1 stabilization—as mediated by extracellular Mg²⁺—and stress the importance of Mg²⁺ status assessment in patients receiving such therapies.

Notably, sensitivity of EM CD8⁺ T cells for Mg²⁺ was lost in the context of supra-physiologic activation, achieved by injection of a secondary anti-antibody crosslinking the CD3/28 targeting mAbs (FIG. 4a). In line, increasing strength of TCR-stimulation—by augmenting concentrations of anti-CD3 antibodies—resulted in less pronounced dependence of PHA T cell blasts on extracellular Mg²⁺ (FIG. 4b) suggesting that Mg²⁺ finetunes activation of LFA-1^{high} T cells in the context of moderate/physiologic stimulation. Using OVA variant peptides (affinity for OT-I: G4<H7<R7), the importance of the Mg²⁺—LFA-1 system in regulating specific target cell lysis was confirmed across a spectrum of TCR affinities, (FIG. 4c). These data further confirmed that cell lysis required Mg²⁺—LFA-1 regulatory function. This was further confirmed by in vitro cytotoxicity assay with polyclonal WT and LFA-1^{-/-} CTLs: While low PHA concentrations (1 μg ml⁻¹) resulted in no cytotoxic activity in neither condition, intermediate PHA concentrations (10 μg ml⁻¹) showed a Mg²⁺ dependent effect in WT CTLs while LFA-1^{-/-} CTLs exhibited impaired cytotoxic capacities compared to WT CTL. Interestingly, high PHA concentrations (100 μg ml⁻¹) equalized the differences between±extracellular Mg²⁺ and LFA-1 genotypes resulting in overall high cytolytic effector functions (FIG. 4d). Moreover, using LFA-1 activating monoclonal antibodies rendered REP T cells independent from extracellular Mg²⁺ concentrations but came at cost of unspecific cytotoxicity (FIG. 4e).

These results suggested that (i) extracellular Mg²⁺ via its moderate LFA-1 stabilizing properties amplified low to intermediate TCR stimuli on LFA-1^{high} T cells resulting in superior T cell activation and subsequent cytotoxic effector functions compared to LFA-1^{-/-} T cells, (ii) using strong LFA-1 stabilizer, such as antibodies, resulted in unspecific target cell killing. Therefore, moderate stabilization of LFA-1—mediated by e.g. Mg²⁺—was indispensable for physiological T cell activation and could directly inform novel therapeutic strategies.

1. A composition for use in cancer immunotherapy comprising
(a) an immune system modulator, wherein the immune system modulator enhances the immune response against cancer, and
(b) an LFA-1 signalling mediator with moderate LFA-1 stabilization properties wherein the LFA-1 signalling mediator significantly enhances the anti-cancer immune response.

2. The composition for use of claim 1, wherein the LFA-1 signalling mediator induces selective T-cell mediated killing of cells presenting tumor-associated antigens.

3. The composition for use of claim 1 or 2, wherein the LFA-1 signalling mediator with moderate LFA-1 stabilization properties induces less T-cell mediated killing of cells not presenting tumor-associated antigens than a signalling mediator with strong LFA-1 stabilization properties.

4. The composition for use of claim 3, wherein the LFA-1 signalling mediator with strong LFA-1 stabilization properties is CBR LFA-1/2.

5. The composition for use of claim 1-4 wherein the LFA-1 signalling mediator binds the metal-ion dependent adhesion site.

6. The composition for use of claim 1-5, wherein the LFA-1 signalling mediator is a divalent cation.

7. The composition for use of claim 6, wherein the divalent cation is Mg²⁺

8. The composition for use according to any one of claims 1-7, wherein the immune system modulator is a monoclonal antibody, a modified immune cell or a checkpoint inhibitor (CPI).

9. The composition for use of claim 8, wherein the checkpoint inhibitor is a PD-1/PDL1 inhibitor.

10. The composition for use of claim 9, wherein the PD-1/PD-L1 inhibitor is an inhibitor selected from the group consisting of nivolumab, pembrolizumab, cemiplimab, spartalizumab, atezolizumab, durvalumab and avelumab.

11. The composition for use according to any one of claims 1-10, additionally comprising a carrier for targeted delivery of the LFA-1 signalling mediator.

12. The composition for use of claim 11, wherein the carrier is a membrane-forming molecule.

13. The composition for use of claim 12, wherein the membrane-forming molecule is a capsule-forming lipid.

14. The composition for use according to any one of claims 1-13, wherein the cancer is selected from the group consisting of breast cancer, brain cancer, blood forming organ cancer, cancer of the immune system, prostate cancer, lung cancer, colon cancer, head and neck cancer, skin cancer, ovary cancer, endometrium cancer, cervix cancer, kidney cancer, lung cancer, stomach cancer, small intestine cancer, liver cancer, pancreas cancer, testis cancer, pituitary gland cancer, blood cancer, spleen cancer, gall bladder cancer, bile duct cancer, esophagus cancer, salivary glands cancer, and the thyroid gland cancer.

15. The composition for use according to any one of claims 1-14, wherein the cancer is a solid tumor and wherein the LFA-1 signalling mediator is administered via intra-tumor injection.


SEQUENCE LISTING (7 sequences)

SEQ ID NO: 1 - protein, 9 aa, Artificial Sequence
  NY-ESO-9c peptide
SLLMWITQC

SEQ ID NO: 2 - protein, 8 aa, Artificial Sequence
  OVA257-264 peptide
SIINFEKL

SEQ ID NO: 3 - protein, 8 aa, Artificial Sequence
  OVA257-264 peptide variant R7
SIIQFERL

SEQ ID NO: 4 - protein, 8 aa, Artificial Sequence
  OVA257-264 peptide variant H7
SIIQFEHL

SEQ ID NO: 5 - protein, 8 aa, Artificial Sequence
  OVA257-264 peptide variant G4
SIIGFEKL

SEQ ID NO: 6 - DNA, 20 bp, Homo sapiens
  Hs.Cas9.ITGAL.1.AA
tgcccgactggcactgatag

SEQ ID NO: 7 - DNA, 20 bp, Mus musculus
  Mm.Cas9.ITGAL.1.AB
cacatagttgatggcacgaa